(19) United States
(12) Patent Application Publication
(10) Pub. No.: US 2023/0383297 A1
(43) Pub. Date: Nov. 30, 2023

(54) NOVEL TARGETS FOR REACTIVATION OF PRADER-WILLI SYNDROME-ASSOCIATED GENES

(71) Applicant: Duke University, Durham, NC (US)

(72) Inventors: Charles A. Gersbach, Chapel Hill, NC (US); Joshua B. Black, Durham, NC (US)

(21) Appl. No.: 18/030,745

(22) PCT Filed: Oct. 8, 2021
(86) PCT No.: PCT/US2021/054292

§ 371 Date: Apr. 6, 2023

Related U.S. Application Data
(60) Provisional application No. 63/090,044, filed on Oct. 9, 2020.

(51) Int. Cl. C12N 15/113; C12N 15/11; C12N 9/22

(52) U.S. Cl. CPC C12N 15/1137; C12N 15/11; C12N 9/22; C12N 2310/20

(57) ABSTRACT

Disclosed herein are compositions and methods for inhibiting a gene selected from OGDH, LIPT1, SDHC, and DHRS7B. The inhibitors may be used to activate SNRPN, SPA1, SPA2, or SNORD118, or a combination thereof. The inhibitors may also be used to treat a subject having Prader Willi Syndrome (PWS) or a PWS-like disorder.

### CROSS-REFERENCE TO RELATED APPLICATIONS

This application claims priority to U.S. Provisional Patent Application No. 63/090,044, filed Oct. 9, 2020, which is incorporated herein by reference in its entirety.

### FIELD

This disclosure relates to compositions and methods for inhibiting several genes. Each gene has been discovered as a target for the reactivation of genes within the Prader-Willi Syndrome (PWS) imprinted locus.

### INTRODUCTION

Prader-Willi Syndrome (PWS) is a neuroendocrine and neurobehavioral disorder associated with genetic and epigenetic abnormalities within the 15q11-13 imprinted locus. Individuals with PWS display mild cognitive impairment and develop a false mental state of starvation that causes hyperphagia beginning in childhood, often resulting in extreme obesity unless strict environmental controls are enforced by caregivers to physically limit access to food. Other symptoms include neonatal hypotonia (weak muscles at birth), growth hormone deficiency, intellectual retardation, anxiety, compulsivity, and behavioral disturbances such as tantrums, outbursts, and self-harm.

While the exact genetic basis of PWS remains unclear, patient mutation profiles have implicated a snoRNA cluster SNORD116 downstream of the SNURF-SNRPN open reading frame controlled by a CpG island imprinting center as a likely contributor to the disease etiology. The genes implicated in PWS are typically expressed only from the paternal copy of chromosome 15, while the PWS genes present on the maternal chromosome are epigenetically silenced. Thus, for example, a patient with paternal deletions or mutations within 15q11.2-13 can present with PWS while retaining functional copies of these genes on the maternal allele. Seventy percent of PWS cases are caused by a large 4-5 Mb deletion on the paternal allele. Twenty-five percent of PWS cases are caused by uniparental maternal disomy (UPD) 15, in which two copies of the maternal chromosome are inherited instead of one copy from each parent. Infrequently, PWS is caused by mutations or microdeletions of the PWS imprinting center. Exceedingly rare cases of PWS are caused by paternal microdeletions of PWS critical region genes, including SNORD116.

Thus, the vast majority of individuals with PWS have at least one ‘good’ (unmutated from a DNA sequence perspective) copy of the PWS chromosomal region in every cell. The vast majority of individuals with UPD have two good copies. These genes, however, are silenced due to genomic imprinting. Reactivation of maternal 15q11-13 provides an opportunity for therapeutic intervention to restore expression of PWS-associated genes. Attempts to activate the silenced PWS region maternal genes using small molecules have met challenges. These challenges have primarily been caused by the redundancy of epigenetic regulation, which makes it difficult for a single agent to remove the imprinting, or by undesirable genome-wide effects. For example, treatment of PWS cells with 5-Aza-DC, a compound that inhibits DNA methylation, can reduce DNA methylation at the PWS imprinting center and reactivate maternal gene expression from the PWS locus but also causes DNA demethylation genome-wide. Similarly, an inhibitor of the histone methyltransferase EHMT2 (also known as G9a) was sufficient to reactivate several of the imprinted genes within the maternal 15q11-13 locus. However, it also carries an added risk of off-target activity resulting from the global loss of an enzyme. Small-molecule-based inhibition of epigenetic modifiers, such as with G9a and 5-Aza-DC, can result in altered expression of many other genes and confer significant toxicity.

Currently there is no cure for PWS and no effective treatment for its symptoms of hyperphagia and anxiety. There remains a need for improved and/or additional therapies for treating PWS.

### SUMMARY

In an aspect, the disclosure relates to a composition for treating a subject having Prader Willi Syndrome (PWS) or a PWS-like disorder, the composition comprising an inhibitor of a gene selected from OGDH, LIPT1, SDHC, and DHRS7B.

In a further aspect, the disclosure relates to a composition for activating SNRPN, SPA1, SPA2, or SNORD116, or a combination thereof, the composition comprising an inhibitor of a gene selected from OGDH, LIPT1, SDHC, and DHRS7B.

In some embodiments, the composition reduces expression of the gene selected from OGDH, LIPT1, SDHC, and DHRS7B, or wherein the composition reduces an activity of a protein encoded by the gene selected from OGDH, LIPT1, SDHC, and DHRS7B.

In some embodiments, the inhibitor comprises a small molecule, a polynucleotide, a polypeptide, or a combination thereof. In some embodiments, the polynucleotide comprises an inhibitory nucleic acid selected from an antisense oligonucleotide, siRNA, RNAi, shRNA, LNA, and PNA. In some embodiments, the inhibitory nucleic acid comprises one or more of a modified internucleoside linkage, a modified sugar moiety, and/or a modified nucleobase. In some embodiments, the inhibitor comprises an antibody.

In some embodiments, the inhibitor comprises a DNA Targeting System. In some embodiments, the DNA Targeting System comprises: (a) a zinc finger protein or TALE or DNA binding fusion protein that targets the gene selected from OGDH, LIPT1, SDHC, and DHRS7B; or (b) a CRISPR/Cas9 system that targets the gene selected from OGDH, LIPT1, SDHC, and DHRS7B. In some embodiments, the DNA binding fusion protein comprises a zinc finger protein or TALE, and a polypeptide domain having transcription repression activity and/or nuclease activity. In some embodiments, the polypeptide domain having transcription repression activity comprises KRAB, MECP2, Mad mSIN3 interaction domain (SID), ERF repressor domain (ERD), SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, and/or CTCF. In some embodiments, the polypeptide domain having nuclease activity comprises FokI. In some embodiments, the CRISPR/Cas9 system comprises: (a) a Cas9 protein or a fusion protein comprising the Cas9 protein; and (b) a gRNA targeting the gene selected from OGDH, LIPT1, SDHC, and DHRS7B, or a portion thereof. In some embodiments, the Cas9 protein is a Streptococcus pyogenes Cas9 protein or a Staphylococcus aureus Cas9 protein. In some embodiments, the Streptococcus pyogenes Cas9 protein comprises the polypeptide sequence of SEQ ID NO: 18, and wherein the Staphylococcus aureus Cas9 protein comprises the polypeptide sequence of SEQ ID NO: 19. In some embodiments, the Cas9 protein is nuclease-deficient dCas9 and comprises the polypeptide sequence of SEQ ID NO: 20 or 21 or is encoded by a polynucleotide sequence comprising SEQ ID NO: 22 or 23. In some embodiments, the fusion protein comprises two heterologous polypeptide domains, wherein the first polypeptide domain comprises the Cas9 protein, and wherein the second polypeptide domain has an activity selected from transcription activation activity, transcription repression activity, transcription release factor activity, histone modification activity, nuclease activity, nucleic acid association activity, methylase activity, and demethylase activity. In some embodiments, the second polypeptide domain has transcription repression activity and/or nuclease activity. In some embodiments, the second polypeptide domain comprises KRAB, MECP2, Mad mSIN3 interaction domain (SID), ERF repressor domain (ERD), SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B. CTCF, and/or FokI. In some embodiments, the fusion protein comprises dCas9-KRAB. In some embodiments, the gene is OGDH and the gRNA binds and targets a polynucleotide sequence selected from SEQ ID NOs: 41-44, or wherein the gene is LIPT1 and the gRNA binds and targets a polynucleotide sequence selected from SEQ ID NOs: 45-48, or wherein the gene is SDHC and the gRNA binds and targets a polynucleotide sequence selected from SEQ ID NOs: 49-52, or wherein the gene is DHRS7B and the gRNA binds and targets a polynucleotide sequence selected from SEQ ID NOs: 53-56.

Another aspect of the disclosure provides a guide RNA (gRNA) that binds and targets a polynucleotide sequence selected from SEQ ID NOs: 41-56, a complement thereof, a variant thereof, or fragment thereof, or that comprises a polynucleotide sequence selected from SEQ ID NOs: 57-72, a complement thereof, a variant thereof, or fragment thereof.

Another aspect of the disclosure provides a polynucleotide encoding a composition as detailed herein or at least one component thereof, or a polynucleotide encoding a gRNA as detailed herein. Another aspect of the disclosure provides a vector comprising the polynucleotide. In some embodiments, the vector is a viral vector. In some embodiments, the vector is a retroviral vector, lentiviral vector, adenoviral vector, adeno-associated virus (AAV) vector, synthetic vector, or vector encapsulated within a lipid nanoparticle.

Another aspect of the disclosure provides a pharmaceutical composition comprising a composition as detailed herein or at least one component thereof, a gRNA as detailed herein, a polynucleotide as detailed herein, or a vector as detailed herein, or a combination thereof.

Another aspect of the disclosure provides a method of treating a subject having PWS or a PWS-like disorder, the method comprising administering to the subject the pharmaceutical composition as detailed herein. In some embodiments, the subject has a PWS Type 1 large deletion, a PWS Type 2 large deletion, a PWS imprinting center mutation, PWS uniparental disomy, a PWS microdeletion encompassing SNORD116 but not MAGEL2, a PWS or PWS-like atypical deletion encompassing MAGEL2 but not SNORD116, heterozygous Schaaf-Yang syndrome, or MAGEL2 disorder. In some embodiments, expression of a gene within the maternal copy of the 15q11-13 locus is increased in the subject.

Another aspect of the disclosure provides a method of activating a gene selected from SNRPN, SPA1, SPA2, and SNORD116, or a combination thereof, in a subject in need thereof, the method comprising administering to the subject the pharmaceutical composition as detailed herein.

The disclosure provides for other aspects and embodiments that will be apparent in light of the following detailed description and accompanying figures.

### BRIEF DESCRIPTION OF THE DRAWINGS

FIG. 1A, FIG. 1B, and FIG. 1C show the generation of a SNRPN-2A-GFP reporter cell line. FIG. 1A is schematic representation of the knock-in of a P2A-GFP cassette into exon ten of SNRPN in a human pluripotent stem cell line using Cas9 nuclease and a donor template. FIG. 1B shows imprinting at the 15q11-13 locus imparts monoallelic expression of SNRPN only from the paternal allele. Consequently, paternal SNRPN-2A-GFP cells are GFP-positive while maternal SNRPN-2A-GFP cells are GFP-negative. FIG. 1C shows the mean fluorescence intensity of clonal human induced pluripotent stem cells that were derived from single cells transfected with the SNRPN-2A-GFP construct. Clone genotype was assessed by PCR. As expected, heterozygous clones that harbor one GFP-tagged allele and one wild-type allele display a bimodal distribution in GFP fluorescence, presumably due to a mixture of paternal and maternal insertions. In the heterozygous clones (middle column), the dashed circle around the top set of dots indicates the paternal insertion, while the solid circle around the bottom set of dots indicates the maternal insertion.

FIG. 2A and FIG. 2B show the reactivation of PWS-associated genes through CRISPR-based gene knockout. FIG. 2A is schematic representation of a CRISPR/Cas9 genome-wide knock-out screen to identify genes controlling expression of the SNRPN host transcript at the 15q11-13 PWS-associated locus in human pluripotent stem cells. A matSNPRN-2A-GFP reporter cell line was transduced with the pooled lentiviral gRNA library at MOI=0.5 and sorted for GFP expression via FACS at Day 14 and Day 21. gRNA abundance in each cell bin was measured by deep sequencing and depleted or enriched gRNAs were identified by differential expression analysis. FIG. 2B is a graph showing differential expression analysis of normalized gRNA counts between the GFP-High and GFP-Low cell populations. Data points with arrows indicate false discovery rate (FDR)<0.05 by differential DESeq2 analysis.

### DETAILED DESCRIPTION

Described herein are compositions and methods for activating a gene such as SNRPN, SPA1, SPA2, or SNORD116, or a combination thereof. The compositions and methods include an inhibitor of a gene selected from OGDH, LIPT1, SDHC, and DHRS7B. The compositions and methods may be used to treat Prader-Willi Syndrome (PWS), Prader-Willi-like syndrome, or disorders that would benefit from activation of genes within the PWS locus (15q11-13), such as SNRPN, SPA1, SPA2, or SNORD116, or a combination thereof. A screen across the human genome employing Cas9 identified genes that if knocked out would result in increased gene expression from the maternal allele in the PWS region. Targeted knockout of these genes resulted in activation of silenced imprinted genes in the PWS region. The disclosure thereby provides a new avenue for epigenetic therapy, by identifying target genes for therapeutic modulation by antisense oligonucleotides (ASOs), siRNA, inhibitory antibodies, and DNA Targeting Systems. DNA Targeting Systems that bind to such targeted genes, compositions comprising such DNA Targeting Systems, and methods of using such DNA Targeting Systems, are further disclosed herein.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Binding region” as used herein refers to the region within a target region that is recognized and bound by the CRISPR/Cas-based gene editing system.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The regulatory elements may include, for example a promoter, an enhancer, an initiation codon, a stop codon, or a polyadenylation signal. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be an subject or cell without an agonist as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Correcting”, “gene editing,” and “restoring” as used herein refers to changing a mutant gene that encodes a dysfunctional protein or truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

“Donor DNA”, “donor template,” and “repair template” as used interchangeably herein refers to a double-stranded DNA fragment or molecule that includes at least a portion of the gene of interest. The donor DNA may encode a full-functional protein or a partially functional protein.

“Enhancer” as used herein refers to non-coding DNA sequences containing multiple activator and repressor binding sites. Enhancers range from 200 bp to 1 kb in length and may be either proximal, 5′ upstream to the promoter or within the first intron of the regulated gene, or distal, in introns of neighboring genes or intergenic regions far away from the locus. Through DNA looping, active enhancers contact the promoter dependently of the core DNA binding motif promoter specificity. 4 to 5 enhancers may interact with a promoter. Similarly, enhancers may regulate more than one gene without linkage restriction and may “skip” neighboring genes to regulate more distant ones. Transcriptional regulation may involve elements located in a chromosome different to one where the promoter resides. Proximal enhancers or promoters of neighboring genes may serve as platforms to recruit more distal elements.

“Frameshift” or“frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas9-based gene editing system, then the cellular machinery will repair the break by homologous recombination, which is enhanced several orders of magnitude in the presence of DNA cleavage. When the homologous DNA piece is absent, non-homologous end joining may take place instead.

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed. The regulatory elements may include, for example a promoter, an enhancer, an initiation codon, a stop codon, or a polyadenylation signal.

“Genome editing” or “gene editing” as used herein refers to changing a gene. Genome editing may include correcting or restoring a mutant gene or adding additional mutations. Genome editing may include knocking out a gene, such as a mutant gene or a normal gene. Genome editing may be used to treat disease or, for example, enhance muscle repair, by changing the gene of interest.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (for example, a “fusion protein,” where the two subsequences are encoded by a single nucleic acid sequence).

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“Partially-functional” as used herein describes a protein that is encoded by a mutant gene and has less biological activity than a functional protein but more than a non-functional protein.

“Polynucleotide” or “nucleic acid” or “oligonucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally derived molecule which is capable of conferring, activating or enhancing expression of a nucleic acid in a cell. A promoter may comprise one or more specific transcriptional regulatory sequences to further enhance expression and/or to after the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter. CMV IE promoter, SV40 early promoter or SV40 late promoter, human U6 (hU6) promoter, and CMV IE promoter.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

“Repeat variable diresidue” or “RVD” refers to a pair of adjacent amino acid residues within the DNA recognition motif (also known as “RVD module”), which includes 33-35 amino acids, of the TALE DNA-binding domain. The RVD determines the nucleotide specificity of the RVD module. RVD modules may be combined to produce an RVD array. The “RVD array length” refers to the number of RVD modules that corresponds to the length of the nucleotide sequence within the target region that is recognized by the binding region of the TALE.

“Sample” or “test sample” as used herein can mean any sample in which the presence and/or level of a target is to be detected or determined or any sample comprising a DNA targeting or gene editing system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamsters, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, or an infant. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Target gene” as used herein refers to any nucleotide sequence encoding a known or putative gene product. The target gene may be a mutated gene involved in a genetic disease. In certain embodiments, the target gene is within or near the 15q11-q13 locus. In certain embodiments, the target gene is a gene selected from OGDH, LIPT1, SDHC, and DHRS7B.

“Target region” as used herein refers to the region of the target gene to which the CRISPR/Cas9-based gene editing or DNA targeting system is designed to bind.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Treatment” or “treating” or “treatment” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Preventing the disease involves administering a composition as disclosed herein to a subject prior to onset of the disease. Suppressing the disease involves administering a composition as disclosed herein to a subject after induction of the disease but before its clinical appearance. Repressing or ameliorating the disease involves administering a composition as disclosed herein to a subject after clinical appearance of the disease.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome, plasmid, or yeast artificial chromosome. A vector may be a DNA or RNA vector. A vector may be a self-replicating extrachromosomal vector. A vector may be an adeno-associated virus (AAV) vector. The vector may encode a Cas9 protein and at least one gRNA molecule.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Prader-Willi Syndrome (PWS)

Provided herein are compositions and methods for treating a subject having Prader Willi Syndrome (PWS) or a PWS-like disorder. Prader-Willi Syndrome (PWS) is a rare genetic disease with a prevalence ranging from approximately one in 8,000 to one in 25,000 patients in the U.S. It is believed that the genetics underlying PWS involve a loss of function of one or more genes on chromosome 15 in humans, in particular, within the PWS region 15q11-13. The 15q11-13 locus may be referred to as the PWS-associated locus or the PWS locus. Loss of function in individuals with PWS can be caused by a de novo deletion in the paternally inherited chromosome (˜70-75%), maternal uniparental disomy (UPD) (˜20-30%), and/or microdeletions or epimutations of the imprinting center, which may be referred to as imprinting defects (˜2-5%).

There are several imprinted genes within the 15q11-13 locus, including the paternally-expressed coding genes MAGEL2, NDN, SNURF-SNRPN, and MKRN3, along with numerous noncoding RNAs (ncRNAs) including the snoRNA clusters SNORD115 and SNORD116. As noted above, PWS patient genotypes most commonly consist of deletions within 15q11-13 that encompass both coding and noncoding genes, although a rare subset of genotypes emphasize the snoRNA clusters as having particular influence in the etiology of PWS. Further evidence suggests that SNURF-SNRPN and downstream ncRNAs, including SPA RNAs and snoRNAs, are processed from a single host transcript that initiates at the imprinting center located in upstream exon 1 of SNRPN.

Other known genes and gene products of the PWS-associated locus may include the following: NPAP1 (NCBI gene ID: 23742), SNORD107 (snoRNA) (NCBI gene ID: 91380), SNORD64 (snoRNA cluster) (NCBI gene ID: 347686), SNORD109A (snoRNA) (NCBI gene ID: 338428), SNORD116 or SNORD116@ (snoRNA gene cluster) (NCBI gene ID: 692236), SPA1 (long noncoding RNA transcribed from the SNORD116 gene cluster), SPA2 (long noncoding RNA transcribed from the SNORD116 gene cluster) (for SPA1 and SPA2, see Wu et al., Mol. Cell 64(3): 534-48 (2016)), 116HG (long non-coding RNA transcribed from SNORD116 gene cluster) (Kocher et al. Genes 2017, 8, 358), SNORD116-1 to 30 (snoRNAs or processed snoRNA derivatives transcribed from the SNORD116 cluster) (SNORD116 1-30 NCBI gene ID Nos: 100033413, 100033414, 100033415, 00033416, 100033417, 100033418, 100033419, 100033420, 100033421, 100033422, 100033423, 100033424, 100033425, 100033426, 100033427, 100033428, 100033429, 100033430, 727708, 100033431, 100033432, 100033433, 100033434, 100033435, 100033436, 100033438, 100033439, 100033820, and 100033821, respectively), SNORD116-30: 100873856, Sno-Inc RNA 1 to 5 (long non coding RNA with snoRNA ends transcribed from the SNORD116 cluster) (Yin et al. Mol. Cell 2012, 48. 219-230), IPW (long noncoding RNA) (NCBI gene ID: 3653), SNORD115 or SNORD115@ (noncoding snoRNA cluster) (NCBI gene ID: 493919), 115HG (long noncoding snoRNA transcribed from SNORD115 cluster) (Powell et al. Hum. Molec. Genet. 2012, 22, 4318-4328), SNORD115-1 to 48 (snoRNAs or processed snoRNA derivates transcribed from SNORD115 cluster) (SNORD115 1-48 NCBI gene ID Nos: 338433, 100033437, 100033440, 100033441, 100033442, 100033443, 100033444, 100033445, 100033446, 100033447, 100033448, 100033449, 100033450, 100033451, 100033453, 100033454, 100033455, 100033456, 100033458, 100033460, 100033603, 100033799, 100033800, 100036563, 100033801, 100033802, 100036564, 100036565, 100033803, 100033804, 100033805, 100033806, 100033807, 100033808, 100033809, 100033810, 100033811, 100033812, 100033813, 100033814, 100033815, 100033816, 100033817, 100033818, 100036566, 100873857, 100036567, 100033822, or SNORD109B (snoRNA) (NCBI gene ID: 338429), and SNHG14 (PWS region long transcript) (NCBI gene ID: 104472715).

PWS-like syndromes and disorders may include but are not limited to Scaaf Yang Syndrome (SYS), Chitayat-Hall Syndrome, Magel2 related disorders, and deletions encompassing Magel2, but not SNORD116. Schaaf-Yang Syndrome (SYS) or MAGEL2-related disorder is a disorder caused by paternally inherited truncating mutations in the MAGEL2 gene. Chitayat-Hall syndrome can also be cause by paternally inherited truncating mutations in the MAGEL2 gene. The compositions and methods detailed herein may be used to treat Prader-Wlli Syndrome (PWS), PWS-like syndrome, PWS Type 1 large deletion, PWS Type 2 large deletion, PWS imprinting center mutation or PWS uniparental disomy; PWS microdeletion, atypical deletion encompassing MAGEL2, Heterozygous Schaaf-Yang syndrome, Chitayat-Hall syndrome, MAGEL2 disorder, and/or MAGEL2-related disorder.

MAGEL2 is a maternally imprinted gene in the PWS region. Patients with Schaaf-Yang syndrome (SYS) display many overlapping symptoms as patients with PWS, including neonatal hypotonia, feeding difficulties during infancy, global developmental delay, and intellectual disabilities. However, there are several features that do not overlap with PWS. Individuals with SYS may commonly present with arthrogryposis or joint contractures which have never been reported in PWS. Additionally, people with SYS may have a higher prevalence of Autism spectrum disorder than is observed in people with PWS. MAGEL2 is a monoexonic gene and therefore missense mutations are not subject to nonsense mediated decay. This, along with the additional phenotypes observed in SYS that are not seen in PWS, or in paternal deletions encompassing MAGEL2 but not SNORD116, suggests that the truncated forms of MAGEL2 present in SYS may have dominant negative activity. Although SYS does not completely overlap with PWS, it demonstrates the importance of the loss of function of the MAGEL2 gene to the PWS phenotype.

The compositions and methods detailed herein may activate or increase the expression of a gene within the PWS region 15q11-13. For example, the compositions and methods as detailed herein may activate or increase the expression of a gene selected from SNRPN, SPA1. SPA2, or SNORD116, or a combination thereof. In some embodiments, the compositions and methods as detailed herein restore expression of maternal genes within the 15q11-13 locus, such as a gene selected from SNRPN. SPA1, SPA2, or SNORD116, or a combination thereof, thereby reintroducing expression lost from the paternal allele. Following treatment with an inhibitor or composition as detailed herein, expression of such maternal genes within the 15q11-13 locus can be restored in PWS patient cells in vitro. Genetically corrected patient cells may be transplanted into a subject. By activating or increasing the expression of a gene selected from SNRPN, SPA1, SPA2, or SNORD116, or a combination thereof, a subject having Prader Willi Syndrome (PWS) or a PWS-like disorder may thereby be treated.

The compositions and methods as described herein may result in amelioration/reduction of PWS symptoms including hypotonia, growth hormone deficiency, infantile failure to thrive, global developmental delay, neonatal hypophagia, anxiety, obsessive compulsive disorder, obsessive compulsive-like disorder, intellectual impairment, intellectual disability, hyperphagia, obesity due to hyperphagia, metabolic syndrome secondary to obesity, type 2 diabetes in PWS, behavioral disturbances such as tantrums, outbursts and self-harm, anxiety and compulsivity, and/or skin picking. Other characteristics or symptoms that may be treated by the compositions and methods detailed herein include small hands, small feet, straight ulnar borders on hands, characteristic facial features such as almond shaped eyes and thin upper lip, temperature instability, chronic constipation, decreased gut/intestinal motility, scoliosis, hyperghrelinemia, and/or hypoinsulinemia.

The compositions and methods as described herein may result in amelioration/reduction of SYS symptoms including neonatal hypotonia, growth hormone deficiency, infantile failure to thrive, global developmental delay, hyperghrelinemia, autism spectrum disorder, infantile respiratory distress, gastroesophageal reflux, chronic constipation, skeletal abnormalities, sleep apnea, temperature instability, and/or arthrogryposis.

### 3. Inhibitors

Provided herein is an inhibitor of a gene selected from OGDH, LIPT1, SDHC, and DHRS7B. The inhibitor may be specific for the gene selected from OGDH, LIPT1, SDHC, and DHRS7B, or a regulatory element thereof, such as, for example, a promoter of the gene. As indicated above, the inhibitor may activate a gene within the PWS region 15q11-13, such as a gene selected from SNRPN, SPA1, SPA2, and SNORD116, or a combination thereof.

In some embodiments, the inhibitor reduces expression of the gene selected from OGDH, LIPT1, SDHC, and DHRS7B. For example, expression of the gene may be reduced at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. Expression of the gene may be knocked out, that is, expression may be reduced 100%. Expression of the gene may be reduced less than about 100%, less than about 95%, less than about 90%, less than about 85%, less than about 80%, less than about 75%, less than about 70%, less than about 65%, less than about 60%, less than about 55%, or less than about 50%.

In some embodiments, the inhibitor reduces an activity of a protein encoded by the gene selected from OGDH, LIPT1, SDHC, and DHRS7B. For example, activity of a protein encoded by the gene may be reduced at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. Activity of a protein encoded by the gene may be knocked out, that is, activity may be reduced 100%. Activity of a protein encoded by the gene may be reduced less than about 100%, less than about 95%, less than about 90%, less than about 85%, less than about 80%, less than about 75%, less than about 70%, less than about 65%, less than about 60%, less than about 55%, or less than about 50%.

The inhibitor may include a small molecule, a polynucleotide, a polypeptide, or a combination thereof. In some embodiments, the inhibitor comprises a polynucleotide. Inhibitors comprising polynucleotides may be referred to as inhibitory nucleic acids. Polynucleotides may include, for example, antisense oligonucleotides (ASOs) or polynucleotides, ribozymes, short hairpin RNA (shRNA), siRNA, single-stranded or double-stranded RNA interference (RNAi), modified bases/locked nucleic acids (LNAs), peptide nucleic acids (PNAs), and/or other oligomeric or oligonucleotides. See, for example, inhibitory nucleic acids disclosed in U.S. Patent Publication No. 2020/0216549, incorporated herein by reference. The polynucleotide may hybridize to at least a portion of a target nucleic acid, such as a gene selected from OGDH, LIPT1, SDHC, and DHRS7B, or a regulatory portion thereof, or a transcribed portion thereof. Binding of the polynucleotide to the target nucleic acid may inhibit the function of the target nucleic acid.

In some embodiments, the polynucleotide is an antisense polynucleotide. Antisense polynucleotides may also be referred to as antisense oligonucleotides. Antisense polynucleotides are typically designed to block expression of a DNA or RNA target by binding to the target and halting expression at the level of transcription, translation, or splicing. Antisense polynucleotides are complementary nucleic acid sequences designed to hybridize under stringent conditions to an RNA. Polynucleotides may be chosen that are sufficiently complementary to the target in that they hybridize sufficiently well and with sufficient specificity to give the desired effect.

In some embodiments, the polynucleotide complementary to a target RNA is an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (for example, each strand comprises a nucleotide sequence that is complementary to a nucleotide sequence in the other strand, such as where the antisense strand and the sense strand form a duplex or double stranded structure); the antisense strand comprises a nucleotide sequence that is complementary to a nucleotide sequence in a target nucleic acid molecule or a portion thereof, and the sense strand comprises nucleotide sequence corresponding to the target nucleic acid sequence or a portion thereof. As another example, interfering RNA may be assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin, or asymmetric hairpin secondary structure, having self-complementary sense and antisense regions, wherein the antisense region comprises a nucleotide sequence that is complementary to a nucleotide sequence in a separate target nucleic acid molecule or a portion thereof and the sense region has a nucleotide sequence corresponding to the target nucleic acid sequence or a portion thereof. The interfering RNA can be a circular single-stranded polynucleotide having two or more loop structures and a stem comprising self-complementary sense and antisense regions, wherein the antisense region comprises a nucleotide sequence that is complementary to a nucleotide sequence in a target nucleic acid molecule or a portion thereof, and the sense region has a nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region, and a loop region. Such an RNA molecule when expressed desirably forms a “hairpin” structure and may be referred to as an “shRNA.” The loop region may generally be between about 2 and about 10 nucleotides in length, or from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. See, for example, Brummelkamp et al. Science 2002, 296, 550-553; Lee et al. Nature Biotechnol. 2002, 20, 500-505; Miyagishi and Taira, Nature Biotechnol. 2002, 20, 497-500; Paddison et al. Genes 8 Dev. 2002, 16, 948-958; Paul, Nature Biotechnol, 2002, 20, 505-508; Sui, PNAS 2002, 99, 5515-5520; Yu et al. PNAS 2002, 99, 6047-6052.

The target RNA cleavage reaction guided by siRNAs may be highly sequence specific. In general, a siRNA containing a nucleotide sequence identical to a portion of the target nucleic acid may be preferred for inhibition. However, 100% sequence identity between the siRNA and the target gene may not be required. Sequence variations due to genetic mutation, strain polymorphism, or evolutionary divergence, for example, may be tolerated. For example, siRNA sequences with insertions, deletions, and single point mutations relative to the target sequence may be effective for inhibition. siRNA sequences with nucleotide analog substitutions or insertions may be effective for inhibition. siRNAs may retain specificity for their target, that is, they may not directly bind to, or directly significantly affect expression levels of, transcripts other than the intended target.

In some embodiments, the inhibitor is a ribozyme. Trans-cleaving enzymatic nucleic acid molecules such as ribozymes can be used and have shown promise as therapeutic agents for human disease (Usman & McSwiggen, Ann. Rep. Med. Chem. 1995, 30, 285-294; Christoffersen and Marr. J. Med. Chem. 1995, 38, 2023-2037). Enzymatic nucleic acid molecules can be designed to cleave specific RNA targets within the background of cellular RNA. Such a cleavage event can render the RNA non-functional.

In general, enzymatic nucleic acids with RNA cleaving activity act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA may destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, Proc. R. Soc. London, B 1979, 205, 435) have been used to evolve new nucleic acid catalysts capable of catalyzing a variety of reactions, such as cleavage and ligation of phosphodiester linkages and amide linkages (Joyce, Gene 1989, 82, 83-87; Beaudry et al. Science 1992, 257, 635-641; Joyce, Scientific American 1992, 267, 90-97; Breaker et al. TIBTECH 1994, 12, 268; Bartel et al. Science 1993, 261, 1411-1418; Szostak, TIBS 1993, 17, 89-93; Kumar et al. FASEB J. 1995, 9, 1183; Breaker, Curr. Op. Biotech. 1996, 1, 442). Ribozymes may be developed to optimize catalytic activity and contribute to any strategy that employs RNA-cleaving ribozymes for the purpose of regulating gene expression, such as, for example, the hammerhead ribozyme, modified hammerhead ribozymes, and other artificial “RNA ligase” ribozymes.

In some embodiments, the polynucleotide is modified. For example, the polynucleotide may be modified to include one or more modified bonds or bases. A number of modified bases may include phosphorothioate, methylphosphonate, peptide nucleic acids, or locked nucleic acid (LNA) molecules. A polynucleotide may be fully modified, while others may be chimeric and contain two or more chemically distinct regions, each made up of at least one nucleotide. These inhibitory nucleic acids may contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids may be formed as composite structures of two or more oligonucleotides, modified oligonucleotides, oligonucleosides, and/or oligonucleotide mimetics as described above. Such chimeric inhibitory nucleic acids may be referred to as hybrids or gapmers. In some embodiments, the polynucleotide is a gapmer, which contains a central stretch (gap) of DNA monomers sufficiently long to induce RNase H cleavage, flanked by blocks of LNA modified nucleotides (see, for example. Stanton et al. Nucleic Acid Ther. 2012, 22, 344-359; Nowotny et al. Cell, 2005, 121, 1005-1016; Kurreck, European Journal of Biochemistry 2003, 270, 1628-1644; Fluiter et al., Mol. Biosyst. 2009, 5, 838-843; incorporated herein by reference). In some embodiments, the polynucleotide is a mixmer, which includes alternating short stretches of LNA and DNA (see, for example, Naguibneva et al., Biomed Pharmacother. 2006, 60, 633-638; Orom et al. Gene 2006, 372, 137-141; incorporated herein by reference). Representative United States patents that disclose the preparation of such hybrid structures may include U.S. Pat. Nos. 5,013,830; 5,149,797; 5,220,007; 5,258,775; 5,366,878; 5,403,711; 5,491,133; 5,565,350; 5,623,065; 5,652,355; 5,652,356; and 5,700,922, each of which is incorporated herein by reference.

In some embodiments, the modified polynucleotide comprises at least one nucleotide modified at the 2′ position of the sugar, such as a 2′-O-alkyl, 2′-O-alkyl-O-alkyl, or 2′-fluoro-modified nucleotide. In other embodiments, RNA modifications include 2′-fluoro, 2′-amino, and 2′ O-methyl modifications on the ribose of pyrimidines, abasic residues, or an inverted base at the 3′ end of the RNA. Such modifications are routinely incorporated into oligonucleotides, and these oligonucleotides have been shown to have a higher Tm (i.e., higher target binding affinity) than 2′-deoxyoligonucleotides against a given target.

A number of nucleotide and nucleoside modifications have been shown to make the oligonucleotide into which they are incorporated more resistant to nuclease digestion than the native oligodeoxynucleotide. These modified polynucleotides may survive intact for a longer period of time than unmodified polynucleotides. Specific examples of modified polynucleotides may include those comprising modified backbones, for example, phosphorothioates, phosphotriesters, methyl phosphonates, short chain alkyl or cycloalkyl intersugar linkages, or short chain heteroatomic or heterocyclic intersugar linkages. Modified polynucleotides may also include phosphorothioate backbones and those with heteroatom backbones, particularly CH2-NH—O—CH2, CH, —N(CH3)-O—CH2 (known as a methylene(methylimino) or MMI backbone), CH2-O—N(CH3)-CH2, CH2-N(CH3)-N(CH3)-CH2, and O—N(CH3)-CH2-CH2 backbones, wherein the native phosphodiester backbone is represented as O—P—O—CH); amide backbones (see, for example, De Mesmaeker et al. Ace. Chem. Res. 1995, 28, 366-374); morpholino backbone structures (see, for example, Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone; see, for example. Nielsen et al., Science 1991, 254, 1497), all references incorporated herein by reference. Phosphorus-containing linkages may include, but are not limited to, phosphorothioates, chiral phosphorothioates, phosphorodithioates, phosphotriesters, aminoalkylphosphotriesters, methyl, and other alkyl phosphonates comprising 3′-alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′ (see, for example, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, incorporated herein by reference). Morpholino-based oligomeric compounds are described in Dwaine A. Braasch and David R. Corey, Biochemistry 2002, 41, 4503-4510); Genesis, volume 30, issue 3, 2001; Heasman, J., Dev. Biol. 2002, 243, 209-214; Nasevicius et al. Nat. Genet. 2000, 26, 216-220; Lacerra et al. Proc. Nad. Acad. Sci. 2000, 97, 9591-9596; and U.S. Pat. No. 5,034,506, all incorporated herein by reference. Cyclohexenyl nucleic acid oligonucleotide mimetics are described in Wang et al. J. Am. Chem. Soc. 2000, 122, 8595-8602, incorporated herein by reference.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These may comprise those having morpholino linkages, formed in part from the sugar portion of a nucleoside; siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH2 component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,833,360; 5,677,437; and 5,677,439, each of which is herein incorporated by reference.

One or more substituted sugar moieties can also be included, for example, one of the following at the 2′ position: OH, SH, SCH3, F, OCN, OCH3 OCH3, OCH3 O(CH2)n CH3, O(CH2)n NH2 or O(CH2)n CH3 where n is from 1 to about 10; C1 to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A modification may include 2′-methoxyethoxy [2′-O—CH2CH2OCH3, also known as 2′-O-(2-methoxyethyl)](Martin et al. Helv. Chim. Acta. 1995, 78, 486). Other modifications may include 2′-methoxy (2′-O—CH3), 2′-propoxy (2′-OCH2CH2CH3) and 2′-fluoro (2′-F). Similar modifications may also be made at other positions on the oligonucleotide, such as the 3′ position of the sugar on the 3 terminal nucleotide and the 5′ position of 5′ terminal nucleotide. Oligonucleotides may also have sugar mimetics such as cyclobutyls in place of the pentofuranosyl group.

Polynucleotides can include, additionally or alternatively, one or more nucleobase modifications or substitutions. As used herein, “unmodified” or “natural” nucleobases comprise the purine bases adenine (A) and guanine (G), and the pyrimidine bases thymine (T), cytosine (C), and uracil (U). Modified nucleobases may include nucleobases found only infrequently or transiently in natural nucleic acids, such as hypoxanthine, 6-methyladenine, 5-Me pyrimidines, 5-methylcytosine (also referred to as 5-methyl-2′ deoxycytosine and 5-Me-C), 5-hydroxymethylcytosine (HMC), glycosyl HMC, gentobiosyl HMC. Modified nucleobases may also include synthetic nucleobases, such as 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine, 2,6-diaminopurine, xanthine, hypoxanthine, 6-methyl and other alkyl derivatives of adenine and guanine, 2-propyl and other alkyl derivatives of adenine and guanine, 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine (see, for example, Komberg, DNA Replication, W. H. Freeman & Co., San Francisco, 1980, pp 75-77; Gebeyehu, G., et al. Nuci. Acids Res. 1987, 15, 4513). A “universal” base known in the art, such as inosine, can also be included. 5-Me-C substitutions may also be included and have been shown to increase nucleic acid duplex stability by 0.6-1.2<0>C. (Sanghvi. Y. S., in Crooke, S. T. and Lebleu, B., eds., Antisense Research and Applications, CRC Press, Boca Raton, 1993, pp. 276-278). Some nucleobases may be useful for increasing the binding affinity of the polynucleotides. These may include 5-substituted pyrimidines, 6-azapyrimidines, and N-2, N-6, and 0-6 substituted purines, comprising 2-aminopropyladenine, 5-propynyluracil, and 5-propynylcytosine. 5-methylcytosine substitutions may be combined with 2′-O-methoxyethyl sugar modifications.

It is not necessary for all positions in a given oligonucleotide to be uniformly modified. More than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds include U.S. Pat. Nos. 5,539,082; 5,714,331; and 5,719,262, each of which is herein incorporated by reference. Further teaching of PNA compounds can be found in Nielsen et al. Science 1991, 254, 1497-1500, incorporated herein by reference. Nucleobases are further described in U.S. Pat. No. 3,687,808; ‘The Concise Encyclopedia of Polymer Science And Engineering’, pages 858-859, Kroschwitz, J. I., ed. John Wiley & Sons, 1990; Englisch et al., Angewandle Chemie, International Edition’, 1991, 30, page 613; Sanghvi, Y. S., Chapter 15, Antisense Research and Applications’, pages 289-302, Crooke, S. T.; and Lebleu, B. ea., CRC Press, 1993. Modified nucleobases are also described in U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,750,692; and 5,681,941, each of which is herein incorporated by reference.

In some embodiments, the polynucleotide is chemically linked to one or more moieties or conjugates that enhance the activity, cellular distribution, or cellular uptake of the oligonucleotide. Such moieties may include but are not limited to, lipid moieties such as a cholesterol moiety (Letsinger et al. Proc. Natl. Acad. Sci. USA 1989, 86, 6553-6556), cholic acid (Manoharan et al., Bioorg. Med. Chem. Let., 1994, 4, 1053-1060), a thioether, such as hexyl-S-tritylthiol (Manoharan et al. Ann. N. Y. Acad. Sci. 1992, 660, 306-309; Manoharan et al. Boorg. Med. Chem. Let. 1993, 3, 2765-2770), a thiocholesterol (Oberhauser et al., Nucl. Acids Res. 1992, 20, 533-538), an aliphatic chain, such as dodecandiol or undecyl residues (Kabanov et al. FEBS Lett. 1990, 259, 327-330; Svinarchuk et al. Biochimie. 1993, 75, 49-54), a phospholipid such as di-hexadecyl-rac-glycerol ortriethylammonium 1,2-di-O-hexadecyl-rac-glycero-3-H-phosphonate (Manoharan et al. Tetrahedron Lett. 1995, 36, 3651-3654; Shea et al. Nucl. Acids Res. 1990, 18, 3777-3783), a polyamine or a polyethylene glycol chain (Mancharan et al. Nucleosides & Nucleotides, 1995, 14, 969-973), or adamantane acetic acid (Manoharan et al., Tetrahedron Left. 1995, 36, 3651-3654). a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther. 1996, 277, 923-937), incorporated herein by reference. See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; and 5,688,941, each of which is herein incorporated by reference.

These moieties or conjugates can include conjugate groups covalently bound to functional groups such as primary or secondary hydroxyl groups. Conjugate groups may include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups may include cholesterols, lipids, phospholipids, biotin, phenazine, folate, phenanthridine, anthraquinone, acridine, fluoresceins, rhodamines, coumarins, and dyes. Groups that enhance the pharmacodynamic properties may include groups that improve uptake, enhance resistance to degradation, and/or strengthen sequence-specific hybridization with the target nucleic acid. Groups that enhance the pharmacokinetic properties may include groups that improve uptake, distribution, metabolism or excretion of the inhibitors. Representative conjugate groups are also disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether such as hexyl-5-tritylthiol, a thiocholesterol, an aliphatic chain such as dodecandiol or undecyl residues, a phospholipid such as di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety (see, for example, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,538; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; and 5,688,941, incorporated herein by reference.

In some embodiments, the polynucleotides comprise locked nucleic acid (LNA) molecules, such as those [alpha]-L-LNAs. LNAs comprise ribonucleic acid analogues wherein the ribose ring is “locked” by a methylene bridge between the 2′-oxygen and the 4′-carbon, such as oligonucleotides containing at least one LNA monomer, that is, one 2′-0,4′-C-methylene-.beta.-D-ribofuranosyl nucleotide. LNA bases may form standard Watson-Crick base pairs but the locked configuration increases the rate and stability of the basepairing reaction (Jensen et al., Oligonucleotides, 2004, 14, 130-146, incorporated herein by reference). LNAs may also have increased affinity to base pair with RNA as compared to DNA. These properties may render LNAs especially useful as probes for fluorescence in situ hybridization (FISH) and comparative genomic hybridization, as knockdown tools for miRNAs, and as antisense oligonucleotides to target mRNAs or other RNAs such as the RNAs as described herein.

LNA molecules can include molecules comprising 10-30 nucleotides, or 12-24 nucleotides, such as 12, 13, 14, 15, 16, 17, 18, 19, 20, 21, 22, 23, 24, 25, 26, 27, 28, 29, or 30 nucleotides in each strand. One of the strands may be substantially identical to a target region in the RNA. One of the strands may be at least 80%, at least 85%, at least 90%, at least 95%, or 100% identical to a target region in the RNA. One of the strands may have 3, 2, 1, or 0 mismatched nucleotide(s) relative to a target region in the RNA. The LNA molecules can be chemically synthesized using methods known in the art.

LNA molecules can be designed using any method known in the art; a number of algorithms are known and are commercially available (for example see exiqon.com; You et al., Nuc. Acids. Res. 2006, 34, e60; McTigue et al., Biochemistry 2004, 43, 5388-5405; and Levin et al., Nuc. Acids. Res. 2006, 34, e14; incorporated herein by reference). For example, “gene walk” methods, similar to those used to design antisense oligos, can be used to optimize the inhibitory activity of the LNA; for example, a series of oligonucleotides of 10-30 nucleotides spanning the length of a target RNA can be prepared, followed by testing for activity. Optionally, gaps, such as gaps of 5-10 nucleotides or more, can be left between the LNAs to reduce the number of oligonucleotides synthesized and tested. GC content may be, for example, between about 30-60%. General guidelines for designing LNAs are known in the art; for example, LNA sequences may bind very tightly to other LNA sequences, so it may be preferable to avoid significant complementarity within an LNA. Contiguous runs of more than four LNA residues may be avoided where possible (for example, it may not be possible with very short (such as about 9-10 nt) oligonucleotides). In some embodiments, the LNAs are xylo-LNAs. For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035988; Koshkin et al. Tetrahedron 1998, 54, 3607-3630; Obika et al. Tetrahedron Lett. 1998, 39, 5401-5404; Jepsen et al. Oligonucleotides 2004, 14, 130-146; Kauppinen et al. Drug Disc. Today 2005, 2, 287-290; and Ponting et al. Cell 2009, 136, 629-641, and references cited therein, all incorporated by reference.

In some embodiments, the inhibitor comprises an antisense oligonucleotide, siRNA, RNAi, shRNA, LNA, and/or PNA. In some embodiments, the inhibitor comprises siRNA. In some embodiments, the inhibitor includes a polynucleotide comprising one or more of a modified internucleoside linkage, a modified sugar moiety, and/or a modified nucleobase as detailed herein.

In some embodiments, the inhibitor comprises a small molecule. Small molecule inhibitors may include CPI-813 (CAS 95809-78-2; LSBio, Seattle, WA, catalog no. LS-H7257-5) as an inhibitor of alpha-ketoglutarate dehydrogenase encoded by the ODGH gene. Small molecule inhibitors may also include Atpenin AS (CAS CAS 119509-24-9; Santa Cruz Biotechnology, Dallas, TX, catalog no. sc-202475) as an inhibitor of SDHC.

In some embodiments, the inhibitor comprises an antibody, such as, for example, an antibody that binds to a protein encoded by a gene selected from OGDH, LIPT1, SDHC, and DHRS7B. Alpha-ketoglutarate dehydrogenase is encoded by the ODGH gene. The LIPT1 gene encodes lipoyltransferase 1. The SDHC gene encodes succinate dehydrogenase complex subunit C, also known as succinate dehydrogenase cytochrome b560 subunit. The DHRS7B gene encodes dehydrogenase/reductase (SDR family) member 7B.

In some embodiments, the inhibitor comprises a DNA Targeting System.

### 4. DNA Targeting System

Provided herein are DNA Targeting Systems that inhibit a gene selected from OGDH. LIPT1. SDHC, and DHRS7B. In some embodiments, the DNA Targeting System includes a DNA binding protein. In some embodiments, the DNA Targeting System includes a CRISPR/Cas9 system.

a. DNA Binding Protein

The DNA Targeting System may include a DNA binding protein. The DNA binding protein may comprise a zinc finger protein or a transcription activator-like effector (TALE). The zinc finger protein or TALE may target a gene selected from OGDH, LIPT1, SDHC, and DHRS7B. In some embodiments, the DNA binding protein is a DNA binding fusion protein.

i) Zinc Finger Protein

A zinc finger protein is a protein that includes one or more zinc finger domains. Zinc finger domains are relatively small protein motifs that contain multiple finger-like protrusions that make tandem contacts with their target molecule such as a DNA target molecule. A zinc finger domain may bind one or more zinc ions or other metal ion such as iron, or in some cases a zinc finger domain forms salt bridges to stabilize the finger-like folds. The zinc binding portion of a zinc finger protein may include one or more cysteine residues and/or one or more histidine residues to coordinate the zinc or other metal ion. A zinc finger protein recognizes and binds to a particular DNA sequence via the zinc finger domain. In some embodiments, a zinc finger protein is fused to or includes a nuclease domain and may be referred to as a zinc finger nuclease (ZFN). The nuclease domain may include, for example, the endonuclease FokI. ZFNs may recognize target sites that consist of two zinc-finger binding sites that flank a 5- to 7-base pair (bp) spacer sequence recognized by the endonuclease FokI cleavage domain.

ii) Transcription Activator-Like Effector (TALE)

A TALE is another type of protein that recognizes and binds to a particular DNA sequence. The DNA-binding domain of a TALE includes an array of tandem 33-35 amino acid repeats, also known as RVD modules. Each RVD module specifically recognizes a single base pair of DNA. RVD modules may be arranged in any order to assemble an array that recognizes a defined DNA sequence. The binding specificity of a TALE DNA-binding domain is determined by the RVD array followed by a single truncated repeat of, for example, 20 amino acids. A TALE DNA-binding domain may have an array of 12 to 27 RVD modules, each RVD module recognizing a single base pair of DNA. Specific RVDs have been identified that recognize each of the four possible DNA nucleotides (A, T. C, and G). Because the TALE DNA-binding domains are modular, repeats that recognize the four different DNA nucleotides may be linked together to recognize any particular DNA sequence. These targeted DNA-binding domains may then be combined with catalytic domains to create functional enzymes, including artificial transcription factors and/or nucleases. In some embodiments, a TALE is fused to or includes a nuclease domain and may be referred to as a TALE nuclease (TALEN). The nuclease domain may include, for example, the endonuclease FokI. TALENs may recognize target sites that consist of two TALE DNA-binding sites that flank a 12-bp to 20-bp spacer sequence recognized by the FokI cleavage domain.

iii) DNA Binding Fusion Protein

Additionally or alternatively, a zinc finger protein or TALE can be fused to a polypeptide domain and referred to as a DNA binding fusion protein. The DNA binding fusion protein may act as a synthetic transcription factor. A zinc finger protein or TALE can be fused to a polypeptide domain having epigenetic modifying activity to mediate targeted gene regulation. For example, the DNA binding fusion protein may include a polypeptide domain having transcription repression activity. A DNA binding fusion protein comprising a zinc finger protein or TALE, and a polypeptide domain having transcription repression activity may mediate targeted gene repression. The polypeptide domain having transcription repression activity may comprise Kruppel associated box activity such as a KRAB domain or KRAB, MECP2, ERF repressor domain (ERD), Mad mSIN3 interaction domain (SID) or Mad-SID repressor domain, SID4X repressor domain, Mxil repressor domain, SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, and/or a domain having TATA box binding protein activity, or a combination thereof. In some embodiments, the polypeptide domain having transcription repression activity comprises KRAB.

In other embodiments, the DNA binding fusion protein includes a polypeptide domain having nuclease activity. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases include deoxyribonuclease and ribonuclease. In some embodiments, the polypeptide domain having nuclease activity comprises FokI.

b. CRISPR/Cas9-Based Gene Editing System

The DNA Targeting System may include a CRISPR/Cas9-based gene editing system that targets the gene selected from OGDH. LIPT1, SDHC, and DHRS7B. The CRISPR/Cas9 system may include a Cas9 protein or a fusion protein, and at least one gRNA. The gRNA may target the gene selected from OGDH, LIPT1, SDHC, and DHRS7B, or a portion thereof.

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a “memory” of past exposures. Cas9 forms a complex with the 3′ end of the sgRNA (which may be referred interchangeably herein as “gRNA”), and the protein-RNA pair recognizes its genomic target by complementary base pairing between the 5′ end of the sgRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed sgRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Type II systems have differing PAM requirements.

An engineered form of the Type II effector system of Streptococcus pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general. Provided herein are CRISPR/Cas9-based engineered systems for use in gene editing and treating genetic diseases. The CRISPR/Cas9-based engineered systems can be designed to target any gene, including genes involved in, for example, a genetic disease, aging, tissue regeneration, or wound healing. The CRISPR/Cas9-based gene editing system can include a Cas9 protein or a Cas9 fusion protein.

i) Cas9 Protein

Cas9 protein is an endonuclease that cleaves nucleic acid and is encoded by the CRISPR loci and is involved in the Type II CRISPR system. The Cas9 protein can be from any bacterial or archaea species, including, but not limited to, Streptococcus pyogenes, Staphylococcus aureus (S. aureus), Acidovorax avenae, Actinobacillus pleuropneumoniae, Actinobacillus succinogenes, Actinobacillus suis, Actinomyces sp., cycliphilus denitrifncans, Aminomonas paucivorans, Bacillus cereus, Bacillus smithii, Bacillus thuringiensis, Bacteroides sp., Blastopirellula manna, Bradyrhizobium sp., Brevibacillus laterosporus, Campylobacter col, Campylobacter jejuni, Campylobacter lari, Candidatus Puniceispillum, Clostidium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In certain embodiments, the Cas9 molecule is a Streptococcus pyogenes Cas9 molecule (also referred herein as “SpCas9”). SpCas9 may comprise an amino acid sequence of SEQ ID NO: 18. In certain embodiments, the Cas9 molecule is a Staphylococcus aureus Cas9 molecule (also referred herein as “SaCas9”). SaCas9 may comprise an amino acid sequence of SEQ ID NO: 19.

A Cas9 molecule or a Cas9 fusion protein can interact with one or more gRNA molecule and, in concert with the gRNA molecule(s), can localize to a site which comprises a target domain, and in certain embodiments, a PAM sequence. The Cas9 protein forms a complex with the 3′ end of a gRNA. The ability of a Cas9 molecule or a Cas9 fusion protein to recognize a PAM sequence can be determined, for example, by using a transformation assay as known in the art.

The specificity of the CRISPR-based system may depend on two factors: the target sequence and the protospacer-adjacent motif (PAM). The target sequence is located on the 5′ end of the gRNA and is designed to bond with base pairs on the host DNA at the correct DNA sequence known as the protospacer. By simply exchanging the recognition sequence of the gRNA, the Cas9 protein can be directed to new genomic targets. The PAM sequence is located on the DNA to be altered and is recognized by a Cas9 protein. PAM recognition sequences of the Cas9 protein can be species specific.

In certain embodiments, the ability of a Cas9 molecule or a Cas9 fusion protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 molecules from different bacterial species can recognize different sequence motifs (for example, PAM sequences). A Cas9 molecule of S. pyogenes may recognize the PAM sequence of NRG (5′-NRG-3′, where R is any nucleotide residue, and in some embodiments, R is either A or G, SEQ ID NO: 1). In certain embodiments, a Cas9 molecule of S. pyogenes may naturally prefer and recognize the sequence motif NGG (SEQ ID NO: 2) and directs cleavage of a target nucleic acid sequence 1 to 10, for example, 3 to 5, bp upstream from that sequence. In some embodiments, a Cas9 molecule of S. pyogenes accepts other PAM sequences, such as NAG (SEQ ID NO: 3) in engineered systems (Hsu et al., Nature Biotechnology 2013 doi:10.1038/nbt.2647). In certain embodiments, a Cas9 molecule of S. thermophilus recognizes the sequence motif NGGNG (SEQ ID NO: 4) and/or NNAGAAW (W=A or T) (SEQ ID NO: 5) and directs cleavage of a target nucleic acid sequence 1 to 10, for example, 3 to 5, bp upstream from these sequences. In certain embodiments, a Cas9 molecule of S. mutans recognizes the sequence motif NGG (SEQ ID NO: 2) and/or NAAR (R=A or G) (SEQ ID NO: 6) and directs cleavage of a target nucleic acid sequence 1 to 10, for example, 3 to 5 bp, upstream from this sequence. In certain embodiments, a Cas9 molecule of S. aureus recognizes the sequence motif NNGRR (R=A or G) (SEQ ID NO: 7) and directs cleavage of a target nucleic acid sequence 1 to 10, for example, 3 to 5, bp upstream from that sequence. In certain embodiments, a Cas9 molecule of S. aureus recognizes the sequence motif NNGRRN (R=A or G) (SEQ ID NO: 8) and directs cleavage of a target nucleic acid sequence 1 to 10, for example, 3 to 5, bp upstream from that sequence. In certain embodiments, a Cas9 molecule of S. aureus recognizes the sequence motif NNGRRT (R=A or G) (SEQ ID NO: 9) and directs cleavage of a target nucleic acid sequence 1 to 10, for example, 3 to 5, bp upstream from that sequence. In certain embodiments, a Cas9 molecule of S. aureus recognizes the sequence motif NNGRRV (R=A or G; V=A or C or G; SEQ ID NO: 10) and directs cleavage of a target nucleic acid sequence 1 to 10, for example, 3 to 5, bp upstream from that sequence. A Cas9 molecule derived from Neisseria meningitidis (NmCas9) normally has a native PAM of NNNNGATT (SEQ ID NO: 11), but may have activity across a variety of PAMs, including a highly degenerate NNNNGNNN PAM (SEQ ID NO: 12) (Esvelt et al. Nature Methods 2013 doi:10.1038/nmeth.2681). In the aforementioned embodiments, N can be any nucleotide residue, for example, any of A. G. C, or T. Cas9 molecules can be engineered to alter the PAM specificity of the Cas9 molecule.

In some embodiments, the Cas9 protein recognizes a PAM sequence NGG (SEQ ID NO: 2) or NGA (SEQ ID NO: 13) or NNNRRT (R=A or G; SEQ ID NO: 14) or ATTCCT (SEQ ID NO: 15) or NGAN (SEQ ID NO: 16) or NGNG (SEQ ID NO: 17). In some embodiments, the Cas9 protein is a Cas9 protein of S. aureus and recognizes the sequence motif NNGRR (R=A or G; SEQ ID NO: 7), NNGRRN (R=A or G; SEQ ID NO: 8), NNGRRT (R=A or G; SEQ ID NO: 9), or NNGRRV (R=A or G; V=A or C or G; SEQ ID NO: 10). In the aforementioned embodiments, N can be any nucleotide residue, for example, any of A, G, C. or T.

Additionally or alternatively, a nucleic acid encoding a Cas9 molecule or Cas9 polypeptide may comprise a nuclear localization sequence (NLS). Nuclear localization sequences are known in the art.

In some embodiments, the at least one Cas9 molecule is a mutant Cas9 molecule. The Cas9 protein can be mutated so that the nuclease activity is inactivated. An inactivated Cas9 protein (“iCas9”, also referred to as “dCas9”) with no endonuclease activity has been targeted to genes in bacteria, yeast, and human cells by gRNAs to silence gene expression through steric hindrance. Exemplary mutations with reference to the S. pyogenes Cas9 sequence to inactivate the nuclease activity include: D10A, E762A, H840A, N854A, N863A and/or D986A. A S. pyogenes Cas9 protein with the D10A mutation may comprise an amino acid sequence of SEQ ID NO: 20. A S. pyogenes Cas9 protein with D10A and H849A mutations may comprise an amino acid sequence of SEQ ID NO: 21. Exemplary mutations with reference to the S. aureus Cas9 sequence to inactivate the nuclease activity include D10A and N580A. In certain embodiments, the mutant S. aureus Cas9 molecule comprises a D10A mutation. The nucleotide sequence encoding this mutant S. aureus Cas9 is set forth in SEQ ID NO: 22. In certain embodiments, the mutant S. aureus Cas9 molecule comprises a N580A mutation. The nucleotide sequence encoding this mutant S. aureus Cas9 molecule is set forth in SEQ ID NO: 23.

A polynucleotide encoding a Cas9 molecule can be a synthetic polynucleotide. For example, the synthetic polynucleotide can be chemically modified. The synthetic polynucleotide can be codon optimized, for example, at least one non-common codon or less-common codon has been replaced by a common codon. For example, the synthetic polynucleotide can direct the synthesis of an optimized messenger mRNA, for example, optimized for expression in a mammalian expression system, as described herein. An exemplary codon optimized nucleic acid sequence encoding a Cas9 molecule of S. pyogenes is set forth in SEQ ID NO: 24. Exemplary codon optimized nucleic acid sequences encoding a Cas9 molecule of S. aureus, and optionally containing nuclear localization sequences (NLSs), are set forth in SEQ ID NOs: 25-31. Another exemplary codon optimized nucleic acid sequence encoding a Cas9 molecule of S. aureus comprises the nucleotides 1293-4451 of SEQ ID NO: 32.

ii) Cas9 Fusion Protein

Alternatively or additionally, the CRISPR/Cas9-based gene editing system can include a fusion protein. The fusion protein can comprise two heterologous polypeptide domains. The first polypeptide domain comprises a Cas9 protein or a mutated Cas9 protein. The first polypeptide domain is fused to at least one second polypeptide domain. The second polypeptide domain has a different activity that what is endogenous to Cas9 protein. For example, the second polypeptide domain may have an activity such as transcription activation activity, transcription repression activity, transcription release factor activity, histone modification activity, nuclease activity, nucleic acid association activity, methylase activity, or demethylase activity. In some embodiments, the second polypeptide domain has transcription activation activity. In some embodiments, the second polypeptide domain has transcription repression activity. In some embodiments, the second polypeptide domain comprises a synthetic transcription factor. The second polypeptide domain may be at the C-terminal end of the first polypeptide domain, or at the N-terminal end of the first polypeptide domain, or a combination thereof. The fusion protein may include one second polypeptide domain. The fusion protein may include two of the second polypeptide domains. For example, the fusion protein may include a second polypeptide domain at the N-terminal end of the first polypeptide domain as well as a second polypeptide domain at the C-terminal end of the first polypeptide domain. In other embodiments, the fusion protein may include a single first polypeptide domain and more than one (for example, two or three) second polypeptide domains in tandem.

(1) Transcription Activation Activity

The second polypeptide domain can have transcription activation activity, for example, a transactivation domain. For example, gene expression of endogenous mammalian genes, such as human genes, can be achieved by targeting a fusion protein of a first polypeptide domain, such as dCas9, and a transactivation domain to mammalian promoters via combinations of gRNAs. The transactivation domain can include a VP16 protein, multiple VP16 proteins, such as a VP48 domain or VP64 domain, p65 domain of NF kappa B transcription activator activity, and/or p300. For example, the fusion protein may comprise dCas9-p300. In some embodiments, p300 comprises a polypeptide having the amino acid sequence of SEQ ID NO: 33 or SEQ ID NO: 34. In other embodiments, the fusion protein comprises dCas9-VP64. In other embodiments, the fusion protein comprises VP64-dCas9-VP64. VP64-dCas9-VP64 may comprise a polypeptide having the amino acid sequence of SEQ ID NO: 35, encoded by the polynucleotide of SEQ ID NO: 36.

(2) Transcription Repression Activity

The second polypeptide domain can have transcription repression activity. The second polypeptide domain may comprise a domain having Kruppel associated box activity such as a KRAB domain or KRAB, ERF repressor domain (ERD), Mad mSIN3 interaction domain (SID) or Mad-SID repressor domain, SID4X repressor domain, Mxil repressor domain. SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, MET1, DRM3, ZMET2, CMT1, CMT2, Laminin A, Laminin B, CTCF, or a domain having TATA box binding protein activity. For example, the fusion protein may be dCas9-KRAB. dCas9-KRAB may comprise a polypeptide having the amino acid sequence of SEQ ID NO: 37, encoded by the polynucleotide of SEQ ID NO: 38.

(3) Transcription Release Factor Activity

The second polypeptide domain can have transcription release factor activity. The second polypeptide domain can have eukaryotic release factor 1 (ERF1) activity or eukaryotic release factor 3 (ERF3) activity.

(4) Histone Modification Activity

The second polypeptide domain can have histone modification activity. The second polypeptide domain can have histone deacetylase, histone acetyltransferase, histone demethylase, or histone methyltransferase activity. The histone acetyltransferase may be p300 or CREB-binding protein (CBP) protein, or fragments thereof. For example, the fusion protein may be dCas9-p300. In some embodiments, p300 comprises a polypeptide of SEQ ID NO: 33 or SEQ ID NO: 34.

(5) Nuclease Activity

The second polypeptide domain can have nuclease activity that is different from the nuclease activity of the Cas9 protein. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases include deoxyribonuclease and ribonuclease. In some embodiments, the polypeptide domain having nuclease activity comprises FokI

(6) Nucleic Acid Association Activity

The second polypeptide domain can have nucleic acid association activity or nucleic acid binding protein-DNA-binding domain (DBD). A DBD is an independently folded protein domain that contains at least one motif that recognizes double- or single-stranded DNA. A DBD can recognize a specific DNA sequence (a recognition sequence) or have a general affinity to DNA. A nucleic acid association region may be selected from helix-turn-helix region, leucine zipper region, winged helix region, winged helix-turn-helix region, helix-loop-helix region, immunoglobulin fold, B3 domain, Zinc finger, HMG-box, Wor3 domain, and TAL effector DNA-binding domain.

(7) Methylase Activity

The second polypeptide domain can have methylase activity, which involves transferring a methyl group to DNA, RNA, protein, small molecule, cytosine, or adenine. In some embodiments, the second polypeptide domain includes a DNA methyltransferase.

(8) Demethylase Activity

The second polypeptide domain can have demethylase activity. The second polypeptide domain can include an enzyme that removes methyl (CH3-) groups from nucleic acids, proteins (in particular histones), and other molecules. Alternatively, the second polypeptide can convert the methyl group to hydroxymethylcytosine in a mechanism for demethylating DNA. The second polypeptide can catalyze this reaction. For example, the second polypeptide that catalyzes this reaction can be Teti.

iii) gRNA

The CRISPR/Cas-based gene editing system includes at least one gRNA molecule. For example, the CRISPR/Cas-based gene editing system may include two gRNA molecules. The at least one gRNA molecule can bind and recognize a target region. The gRNA provides the targeting of a CRISPR/Cas9-based gene editing system. The gRNA is a fusion of two noncoding RNAs: a crRNA and a tracrRNA. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to bind, and in some cases, cleave the target nucleic acid. The gRNA may target any desired DNA sequence by exchanging the sequence encoding a 20 bp protospacer which confers targeting specificity through complementary base pairing with the desired DNA target. The “target region” or “target sequence” refers to the region of the target gene to which the CRISPR/Cas9-based gene editing system targets and binds. The portion of the gRNA that targets the target sequence in the genome may be referred to as the “targeting sequence” or “targeting portion” or “targeting domain.” “Protospacer” or “gRNA spacer” may refer to the region of the target gene to which the CRISPR/Cas9-based gene editing system targets and binds; “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to the sequence that the gRNA targets. Together, the gRNA targeting portion and gRNA scaffold form one polynucleotide. The constant region of the gRNA may include the sequence of SEQ ID NO: 39 (RNA), which is encoded by a sequence comprising SEQ ID NO: 40 (DNA). The CRISPR/Cas9-based gene editing system may include at least one gRNA, wherein the gRNAs target different DNA sequences. The target DNA sequences may be overlapping. The target sequence or protospacer is followed by a PAM sequence at the 3′ end of the protospacer in the genome. Different Type II systems have differing PAM requirements, as detailed above.

The DNA-targeting domain of the guide RNA does not need to be perfectly complementary to the target region of the target DNA. In example embodiments, the DNA-targeting domain of the guide RNA sequence is at least 80% complementary, preferably at least 85%, 90%, 95%, 96%, 97%, 98%, or at least 99% complementary to (or has 1, 2 or 3 mismatches compared to) the target region over a length of, such as, 10, 11, 12, 13, 14, 15, 16, 17, 18, 19 or 20 nucleotides. For example, the DNA-targeting domain of the guide RNA sequence is at least 80% complementary over at least 18 nucleotides of the target region. The target region may be on either strand of the target DNA.

The portion of the guide RNA that corresponds to the tracrRNA can be variably truncated and a range of lengths has been shown to function in both a system comprising separate RNAs and a system comprising a single-guide RNA. For example, in some embodiments, tracrRNA may be truncated from its 3′ end by at least 1, 2, 3, 4, 5, 6, 7, 8, 9, 10, 15, 20, 25, 30, 35, or 40 nucleotides. In some embodiments, the tracrRNA may be truncated from its 5′ end by at least 1, 2, 3, 4, 5, 6, 7, 8, 9, 10, 15, 20, 21, 22, 23, 24, or 25 nucleotides. Alternatively, the tracrRNA may be truncated from both the 5′ and 3′ end, such as, by at least 1, 2, 3, 4, 5, 6, 7, 8, 9, 10, 15, or 20 nucleotides on the 5′ end and at least 1, 2, 3, 4, 5, 6, 7, 8, 9, 10, 15, 20, 25, 30, 35, or 40 nucleotides on the 3′ end.

The gRNA may target a gene selected from OGDH, LIPT1, SDHC, and DHRS7B. The gRNA may target a region within or near the gene. For example, the gRNA may target a regulatory element of a gene selected from OGDH, LIPT1, SDHC, and DHRS7B. In some embodiments, the gRNA targets a promoter of a gene selected from OGDH, LIPT1, SDHC, and DHRS7B. The gRNA may bind and target a polynucleotide sequence comprising at least one of SEQ ID NOs: 41-56, or a complement thereof, or a variant thereof, or a truncation thereof. The gRNA may be encoded by a polynucleotide sequence comprising at least one of SEQ ID NOs: 41-56, or a complement thereof, or a variant thereof, or a truncation thereof. The gRNA may comprise a polynucleotide of at least one of SEQ ID NOs: 57-72, or a complement thereof, or a variant thereof, or a truncation thereof. A truncation may be 1, 2, 3, 4, 5, 6, 7, 8, or 9 nucleotides shorter than the sequence of any one of SEQ ID NOs: 41-72 or a complement or a variant thereof.

As described above, the gRNA molecule comprises a targeting domain (also referred to as targeted or targeting sequence), which is a polynucleotide sequence complementary to the target DNA sequence. The gRNA may comprise a “G” at the 5′ end of the targeting domain or complementary polynucleotide sequence. The CRISPR/Cas9-based gene editing system may use gRNAs of varying sequences and lengths. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The number of gRNA molecules that may be included in the CRISPR/Cas9-based gene editing system can be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 18 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA molecules that may be included in the CRISPR/Cas9-based gene editing system can be less than 50 different gRNAs, less than 45 different gRNAs, less than 40 different gRNAs, less than 35 different gRNAs, less than 30 different gRNAs, less than 25 different gRNAs, less than 20 different gRNAs, less than 19 different gRNAs, less than 18 different gRNAs, less than 17 different gRNAs, less than 16 different gRNAs, less than 15 different gRNAs, less than 14 different gRNAs, less than 13 different gRNAs, less than 12 different gRNAs, less than 11 different gRNAs, less than 10 different gRNAs, less than 9 different gRNAs, less than 8 different gRNAs, less than 7 different gRNAs, less than 8 different gRNAs, less than 5 different gRNAs, less than 4 different gRNAs, less than 3 different gRNAs, or less than 2 different gRNAs. The number of gRNAs that may be included in the CRISPR/Cas9-based gene editing system can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

### 5. Genetic Constructs

The inhibitor, DNA Targeting System or at least one component thereof, or gRNA may be encoded by or comprised within a genetic construct. The genetic construct, such as a plasmid or expression vector, may comprise a nucleic acid that encodes the inhibitor, the DNA Targeting System, and/or at least one of the gRNAs. In some embodiments, a genetic construct encodes a zinc finger nuclease (ZFN) or TALE that targets the gene selected from OGDH, LIPT1, SDHC, and DHRS7B. In some embodiments, a genetic construct encodes a CRISPR/Cas9 system that targets the gene selected from OGDH, LIPT1, SDHC, and DHRS7B. In certain embodiments, a genetic construct encodes one gRNA molecule, i.e., a first gRNA molecule, and optionally a Cas9 molecule or fusion protein. In some embodiments, a genetic construct encodes two gRNA molecules, i.e., a first gRNA molecule and a second gRNA molecule, and optionally a Cas9 molecule or fusion protein. In some embodiments, a first genetic construct encodes one gRNA molecule, i.e., a first gRNA molecule, and optionally a Cas9 molecule or fusion protein, and a second genetic construct encodes one gRNA molecule, i.e., a second gRNA molecule, and optionally a Cas9 molecule or fusion protein.

Genetic constructs may include polynucleotides such as vectors and plasmids. The genetic construct may be a linear minichromosome including centromere, telomeres, or plasmids or cosmids. The vector may be an expression vectors or system to produce protein by routine techniques and readily available starting materials including Sambrook et al., Molecular Cloning and Laboratory Manual. Second Ed., Cold Spring Harbor (1989), which is incorporated fully by reference. The construct may be recombinant. The genetic construct may be part of a genome of a recombinant viral vector, including recombinant lentivirus, recombinant adenovirus, and recombinant adenovirus associated virus. The genetic construct may comprise regulatory elements for gene expression of the coding sequences of the nucleic acid. The regulatory elements may include, for example a promoter, an enhancer, an initiation codon, a stop codon, or a polyadenylation signal.

The genetic construct may comprise heterologous nucleic acid encoding the CRISPR/Cas-based gene editing system and may further comprise an initiation codon, which may be upstream of the CRISPR/Cas-based gene editing system coding sequence, and a stop codon, which may be downstream of the CRISPR/Cas-based gene editing system coding sequence. The initiation and termination codon may be in frame with the CRISPR/Cas-based gene editing system coding sequence. The vector may also comprise a promoter that is operably linked to the CRISPR/Cas-based gene editing system coding sequence. The promoter may be a constitutive promoter, an inducible promoter, a repressible promoter, or a regulatable promoter. The promoter may be a ubiquitous promoter. The promoter may be a tissue-specific promoter. The tissue specific promoter may be a muscle specific promoter. The tissue specific promoter may be a skin specific promoter. The CRISPR/Cas-based gene editing system may be under the light-inducible or chemically inducible control to enable the dynamic control of gene/genome editing in space and time. The promoter operably linked to the CRISPR/Cas-based gene editing system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barrvirus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. Examples of a tissue specific promoter, such as a muscle or skin specific promoter, natural or synthetic, are described in U.S. Patent Application Publication No. US20040175727, the contents of which are incorporated herein in its entirety. The promoter may be a CK8 promoter, a Spc512 promoter, a MHCK7 promoter, for example. The promoter may be a neuron-specific promoter. The promoter may be a synapsin promoter.

The genetic construct may also comprise a polyadenylation signal, which may be downstream of the CRISPR/Cas-based gene editing system. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 vector (Invitrogen, San Diego, CA).

Coding sequences in the genetic construct may be optimized for stability and high levels of expression. In some instances, codons are selected to reduce secondary structure formation of the RNA such as that formed due to intramolecular bonding.

The genetic construct may also comprise an enhancer upstream of the CRISPR/Cas-based gene editing system or gRNAs. The enhancer may be necessary for DNA expression. The enhancer may be human actin, human myosin, human hemoglobin, human muscle creatine or a viral enhancer such as one from CMV, HA, RSV, or EBV. Polynucleotide function enhancers are described in U.S. Pat. Nos. 5,593,972, 5,962,428, and WO94/016737, the contents of each are fully incorporated by reference. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered. The genetic construct may also comprise a reporter gene, such as green fluorescent protein (“GFP”) and/or a selectable marker, such as hygromycin (“Hygro”).

The genetic construct may be useful for transfecting cells with nucleic acid encoding the CRISPR/Cas-based gene editing system, which the transformed host cell is cultured and maintained under conditions wherein expression of the CRISPR/Cas-based gene editing system takes place. The genetic construct may be transformed or transduced into a cell. The genetic construct may be formulated into any suitable type of delivery vehicle including, for example, a viral vector, lentiviral expression, mRNA electroporation, and lipid-mediated transfection for delivery into a cell. The genetic construct may be part of the genetic material in attenuated live microorganisms or recombinant microbial vectors which live in cells. The genetic construct may be present in the cell as a functioning extrachromosomal molecule.

Further provided herein is a cell transformed or transduced with a system or component thereof as detailed herein. Suitable cell types are detailed herein. In some embodiments, the cell is a stem cell. The stem cell may be a human stem cell. In some embodiments, the cell is an embryonic stem cell. The stem cell may be a human pluripotent stem cell (iPSCs). Further provided are stem cell-derived neurons, such as neurons derived from iPSCs transformed or transduced with a DNA targeting system or component thereof as detailed herein.

a. Viral Vectors

A genetic construct may be a viral vector. Further provided herein is a viral delivery system. Viral delivery systems may include, for example, lentivirus, retrovirus, adenovirus, mRNA electroporation, synthetic vector, vector encapsulated within a lipid nanoparticle, or nanoparticles. In some embodiments, the vector is a modified lentiviral vector. In some embodiments, the viral vector is an adeno-associated virus (AAV) vector. The AAV vector is a small virus belonging to the genus Dependovirus of the Parvoviridae family that infects humans and some other primate species.

AAV vectors may be used to deliver CRISPR/Cas9-based gene editing systems using various construct configurations. For example, AAV vectors may deliver Cas9 or fusion protein and gRNA expression cassettes on separate vectors or on the same vector. Alternatively, if the small Cas9 proteins or fusion proteins, derived from species such as Staphylococcus aureus or Neisseria meningitidis, are used then both the Cas9 and up to two gRNA expression cassettes may be combined in a single AAV vector. In some embodiments, the AAV vector has a 4.7 kb packaging limit.

In some embodiments, the AAV vector is a modified AAV vector. The modified AAV vector may have enhanced neuron and/or brain tissue tropism. The modified AAV vector may have enhanced cardiac and/or skeletal muscle tissue tropism. The modified AAV vector may be capable of delivering and expressing the CRISPR/Cas9-based gene editing system in the cell of a mammal. For example, the modified AAV vector may be an AAV-SASTG vector (Piacentino et al. Human Gene Therapy 2012, 23, 635-646). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy 2012, 12, 139-151). The modified AAV vector may be AAV2i8G9 (Shen et al. J. Biot. Chem. 2013, 288, 28814-28823).

The genetic construct may comprise a polynucleotide sequence selected from SEQ ID NOs: 41-56, or a complement thereof.

### 6. Pharmaceutical Compositions

Further provided herein are pharmaceutical compositions comprising the above-described inhibitor, DNA Targeting System or at least one component thereof, gRNA, or genetic construct. In some embodiments, the pharmaceutical composition may comprise about 1 ng to about 10 mg of DNA encoding the inhibitor, DNA Targeting System, gRNA, or genetic construct. The systems or genetic constructs as detailed herein, or at least one component thereof, may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate, and more preferably, the poly-L-glutamate may be present in the composition for gene editing in skeletal muscle or cardiac muscle at a concentration less than 6 mg/mL.

### 7. Administration

The inhibitor, DNA Targeting System or at least one component thereof, gRNA, or genetic construct as detailed herein, or pharmaceutical composition comprising the same, may be administered or delivered to a cell. Methods of introducing a nucleic acid into a host cell are known in the art, and any known method can be used to introduce a nucleic acid (e.g., an expression construct) into a cell. Suitable methods include, for example, viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. The inhibitor, DNA Targeting System, gRNA, or genetic construct, or component thereof, or composition comprising the same, may be electroporated using BioRad Gene Pulser Xcell or Amaxa Nucleofector lIb devices or other electroporation device. Several different buffers may be used, including BioRad electroporation solution, Sigma phosphate-buffered saline product #08537 (PBS), Invitrogen OptiMEM I (OM), or Amaxa Nucleofector solution V (N.V.). Transfections may include a transfection reagent, such as Lipofectamine 2000.

The inhibitor, DNA Targeting System or at least one component thereof, gRNA, or genetic construct as detailed herein, or pharmaceutical composition comprising the same, may be administered to a subject. Such compositions can be administered in dosages and by techniques well known to those skilled in the medical arts taking into consideration such factors as the age, sex, weight, and condition of the particular subject, and the route of administration. The presently disclosed inhibitor, DNA Targeting System or at least one component thereof, gRNA, or genetic construct as detailed herein, or pharmaceutical composition comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the inhibitor, DNA Targeting System or at least one component thereof, gRNA, or genetic construct as detailed herein, or pharmaceutical composition comprising the same, is administered to a subject intramusculariy, intravenously, or a combination thereof. The inhibitor, DNA Targeting System or at least one component thereof, gRNA, or genetic construct as detailed herein, or pharmaceutical composition comprising the same may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the brain or other component of the central nervous system. For veterinary use, the systems, genetic constructs, or compositions comprising the same may be administered as a suitably acceptable formulation in accordance with normal veterinary practice. The veterinarian may readily determine the dosing regimen and route of administration that is most appropriate for a particular animal. The inhibitor, DNA Targeting System or at least one component thereof, gRNA, or genetic construct as detailed herein, or pharmaceutical composition comprising the same may be administered by traditional syringes, needleless injection devices, “microprojectile bombardment gone guns,” or other physical methods such as electroporation (“EP”), “hydrodynamic method”, or ultrasound. Alternatively, transient in vivo delivery of CRISPR/Cas-based systems by non-viral or non-integrating viral gene transfer, or by direct delivery of purified proteins and gRNAs containing cell-penetrating motifs may enable highly specific correction and/or restoration in situ with minimal or no risk of exogenous DNA integration.

Upon delivery of the presently disclosed inhibitor, DNA Targeting System or at least one component thereof, gRNA, or genetic construct as detailed herein, or pharmaceutical composition comprising the same, and thereupon the vector into the cells of the subject, the transfected cells may express the inhibitor, the DNA binding protein, the gRNA molecule(s), and/or the Cas9 molecule or fusion protein.

a. Cell Types

Any of the delivery methods and/or routes of administration detailed herein can be utilized with a myriad of cell types, for example, those cell types currently under investigation for cell-based therapies, including, but not limited to, immortalized neuronal cells, such as wild-type and PWS patient derived cell lines, primary neurons in the brain, stem cell-derived neurons, stem cells such as induced pluripotent stem cells, bone marrow-derived progenitors, CD 133+ cells, mesoangioblasts, mesenchymal progenitor cells, and hematopoietic stem cells. Immortalization of human cells can be used for clonal derivation of genetically corrected cells. Cells can be modified ex vivo to isolate and expand clonal populations of immortalized cells that include a genetically corrected or restored gene and are free of other nuclease-introduced mutations in protein coding regions of the genome.

### 8. Kits

Provided herein is a kit, which may be used to activate a gene selected from SNRPN, SPA1, SPA2, or SNORD116, or a combination thereof, or to treat a subject having Prader Willi Syndrome (PWS) or a PWS-like disorder. The kit comprises genetic constructs or a composition comprising the same, as described above, and instructions for using said composition. In some embodiments, the kit comprises at least one gRNA targeting a polynucleotide sequence selected from SEQ ID NOs: 41-56, a complement thereof, a variant thereof, or fragment thereof, or a gRNA comprising a polynucleotide sequence selected from SEQ ID NOs: 57-72, a complement thereof, a variant thereof, or fragment thereof, and instructions for using the CRISPR/Cas-based gene editing system.

Instructions included in kits may be affixed to packaging material or may be included as a package insert. While the instructions are typically written on printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The genetic constructs or a composition comprising the same for activating a gene selected from SNRPN, SPA1, SPA2, or SNORD116, or a combination thereof, or for treating a subject having Prader Willi Syndrome (PWS) or a PWS-like disorder may include a modified AAV vector. The modified AAV vector may include a gRNA molecule(s) and a Cas9 protein or fusion protein, as described above, that specifically binds and cleaves a region of a gene selected from OGDH, LIPT1, SDHC, and DHRS7B.

### 9. Methods

a. Methods of Treating a Subject Having PWS or a PWS-Like Disorder

Provided herein are methods of treating a subject having PWS or a PWS-like disorder. The methods may include administering to the subject an inhibitor of a gene selected from OGDH. LIPT1, SDHC, and DHRS7B, as detailed herein. In some embodiments, the inhibitor comprises a small molecule, a polynucleotide, a polypeptide, siRNA, an antibody, or a DNA Targeting System as detailed herein. In some embodiments, the subject has a PWS Type 1 large deletion, a PWS Type 2 large deletion, a PWS imprinting center mutation, PWS uniparental disomy, a PWS microdeletion encompassing SNORD116 but not MAGEL2, a PWS or PWS-like atypical deletion encompassing MAGEL2 but not SNORD116, heterozygous Schaaf-Yang syndrome, or MAGEL2 disorder. In some embodiments, upon administration of the inhibitor to the subject, expression of a gene within the maternal copy of the 15q11-13 locus is increased in the subject.

b. Methods of Activating a Gene

Provided herein are methods of activating a gene selected from SNRPN, SPA1, SPA2, and SNORD116, or a combination thereof. The methods may include administering to the subject an inhibitor of a gene selected from OGDH, LIPT1, SDHC, and DHRS7B, as detailed herein. In some embodiments, the inhibitor comprises a small molecule, a polynucleotide, a polypeptide, siRNA, an antibody, or a DNA Targeting System as detailed herein. In some embodiments, the subject has a PWS Type 1 large deletion, a PWS Type 2 large deletion, a PWS imprinting center mutation, PWS uniparental disomy, a PWS microdeletion encompassing SNORD116 but not MAGEL2, a PWS or PWS-like atypical deletion encompassing MAGEL2 but not SNORD116, heterozygous Schaaf-Yang syndrome, or MAGEL2 disorder. In some embodiments, upon administration of the inhibitor to the subject, expression of a gene within the maternal copy of the 15q11-13 locus is increased in the subject.

### 10. Examples

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### matSNPRN-2A-GFP Reporter Cell Line

There are several imprinted genes within the 15q11-13 locus, including the paternally-expressed coding genes MAGEL2, NDN, and SNURF-SNRPN, along with numerous noncoding RNAs (ncRNAs), including the snoRNA clusters SNORD115 and SNORD116. SNRPN expression was selected as a proxy for the imprinting status of the 15q11-13 locus.

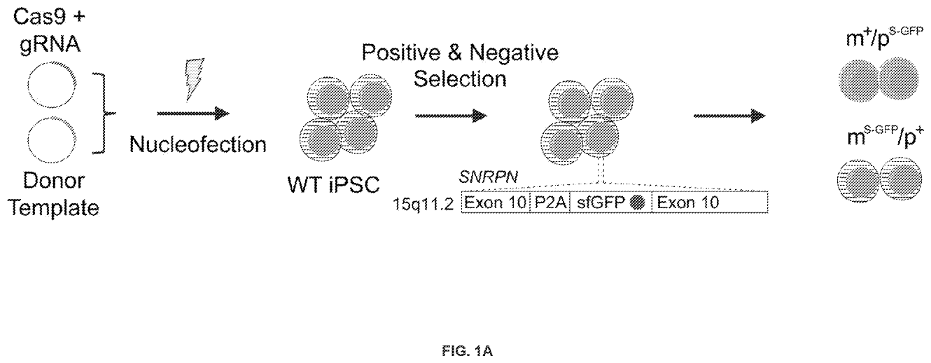

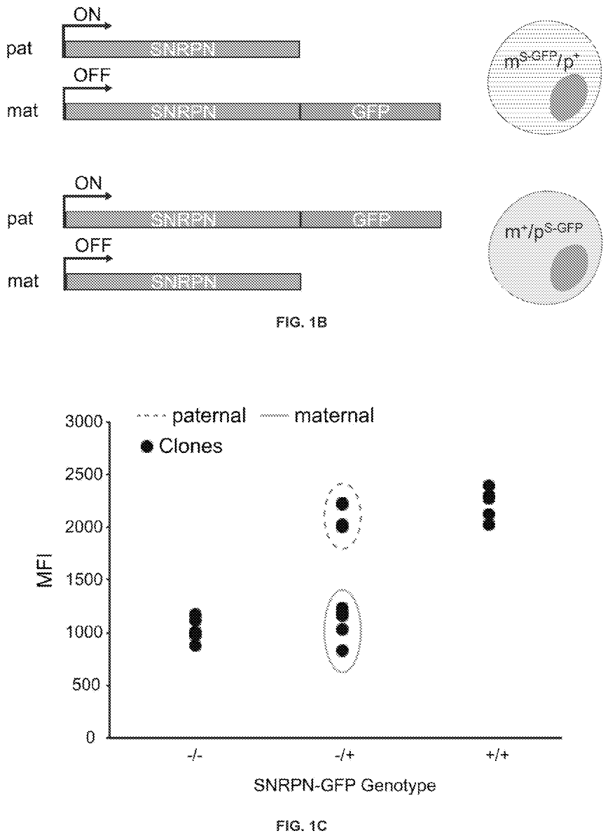

Superfolder GFP (sfGFP) was inserted into exon 10 of SNURF-SNRPN in frame with the SNRPN ORF in a wild-type human pluripotent stem cell line with two intact copies of 15q11-13 (FIG. 1A). A P2A skipping peptide was included between SNPRN and sfGFP to link the expression of the two proteins while avoiding disrupting SNPRN function, localization, or stability with a direct fusion to sfGFP. Heterozygous clones were created with GFP-tagged SNRPN on either the maternal or paternal allele. Only the paternally-tagged cells were GFP-positive, indicating that imprinting is accurately maintained in this cell line (FIG. 1B). As expected, clonal lines were derived that had either the maternal or paternal allele tagged (GFP-tagged SNRPN on), with the heterozygous clones uniquely displaying a bimodal distribution in GFP fluorescence (FIG. 1C). These two SNRPN-2A-GFP lines independently report on SNRPN expression from the paternal or maternal allele, respectively.

### Example 2

### Reactivation of PWS-Associated Genes Through CRISPR-Based Gene Knockout

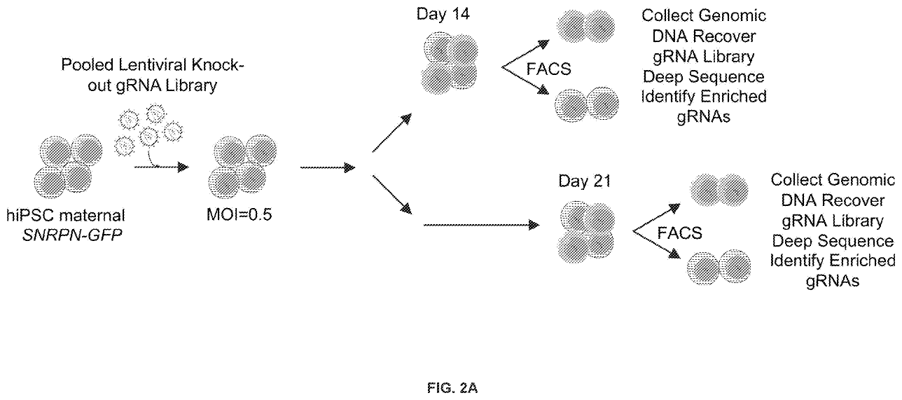

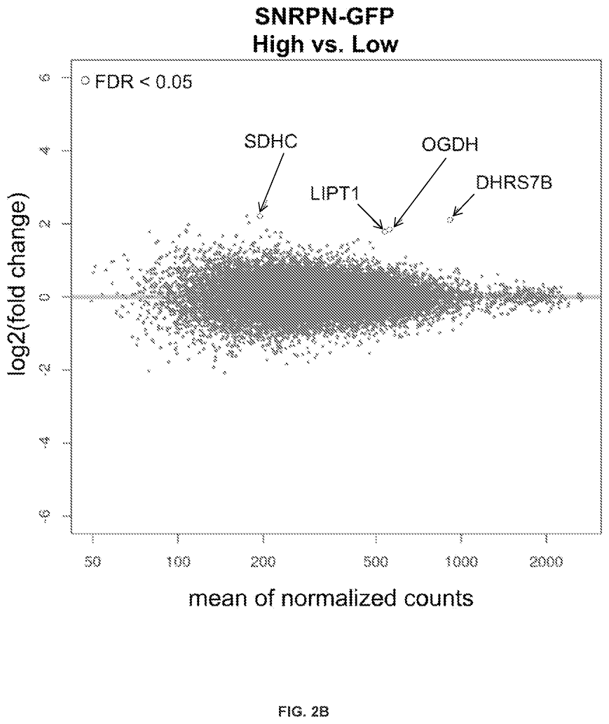

A genome-wide CRISPR/Cas9-based screening approach was used to identify genes whose knock-out results in reactivation of PWS-associated imprinted genes in human cells (FIG. 2A). A SNRPN-2A-GFP cell line that independently reports on SNRPN expression from the maternal allele, as detailed in Example 1, was transfected with lentivirus encoding a library of gRNAs and Cas9. The gRNA library was commercially obtained from Addgene (Watertown, MA; catalog no. 73179) and included approximately 80,000 gRNAs, each gRNA targeting a different gene across the human genome. The matSNPRN-2A-GFP reporter cell line was transduced with the pooled lentiviral gRNA library at a low multiplicity of infection (MOI) of 0.5 to limit the number of gRNAs per cell, and cells were sorted based on GFP expression via FACS at Day 14 and Day 21. gRNA abundance in each cell bin was measured by deep sequencing and depleted or enriched gRNAs were identified by differential expression analysis of gRNA frequencies between the high and low GFP expressing cells. Using the SNRPN-GFP human pluripotent stem cell line, four novel target genes were identified to increase SNRPN-GFP expression following targeted gene knockout, providing new avenues of therapeutic intervention for PWS. The differential expression analysis of normalized gRNA counts between the GFP-High and GFP-Low cell populations is shown in FIG. 2B. The four genes identified as significantly enriched in GFP-High cells (FDR<0.05) include Oxoglutarate Dehydrogenase (OGDH), Lipoyltransferase 1 (LIPT1), Succinate Dehydrogenase Complex Subunit C (SDHC), and Dehydrogenase/Reductase 7B (DHRS7B). These four genes have not previously been linked to regulation of imprinted genes at the 151q1-13 locus. The gRNAs used to target the four genes targeted the DNA sequences shown in TABLE 1.

TABLE 1
Target sequences of the gRNAs.

| Gene | | | |
|---|---|---|---|
| Targeted | gRNA ID | gRNA targeted sequence | gRNA sequence |
| OGDH | OGDH.1 | GACTAGTTCGAACTATGTGG | GACUAGUUCGAACUAUGUGG |
| | | (SEQ ID NO: 41) | (SEQ ID NO: 57) |
| OGDH | OGDH.2 | GATCATGCAGTTCACAAATG | GAUCAUGCAGUUCACAAAUG |
| | | (SEQ ID NO: 42) | (SEQ ID NO: 58) |
| OGDH | OGDH.3 | GTTGGCCACTCATAGATACG | GUUGGCCACUCAUAGAUACG |
| | | (SEQ ID NO: 43) | (SEQ ID NO: 59) |
| OGDH | OGDH.4 | TTCCTGTCCCCCGATGAAAG | UUCCUGUCCCCCGAUGAAAG |
| | | (SEQ ID NO: 44) | (SEQ ID NO: 60) |
| LIPT1 | LIPT1.1 | AGTACTTCACAGGTCAGAGT | AGUACUUCACAGGUCAGAGU |
| | | (SEQ ID NO: 45) | (SEQ ID NO: 61) |
| LIPT1 | LIPT1.2 | ATGCCTACCAATTACAACAG | AUGCCUACCAAUUACAACAG |
| | | (SEQ ID NO: 46) | (SEQ ID NO: 62) |
| LIPT1 | LIPT1.3 | GAACAGCTTCTAAGATCGGC | GAACAGCUUCUAAGAUCGGO |
| | | (SEQ ID NO: 47) | (SEQ ID NO: 63) |
| LIPT1 | LIPT1.4 | GGAACAGTCTACCATGATAT | GGAACAGUCUACCAUGAUAU |
| | | (SEQ ID NO: 48) | (SEQ ID NO: 64) |
| SDHC | SDHC.1 | AAGCAATACCAGTGCCACGG | AAGCAAUACCAGUGCCACGG |
| | | (SEQ ID NO: 49) | (SEQ ID NO: 65) |
| SDHC | SDHC.2 | CGGCCAAAGAAGAGATGGAG | CGGCCAAAGAAGAGAUGGAG |
| | | (SEQ ID NO: 50) | (SEQ ID NO: 66) |
| SDHC | SDHC.3 | GCCAAAAAGAGAGACCCCTG | GCCAAAAAGAGAGACCCCUG |
| | | (SEQ ID NO: 51) | (SEQ ID NO: 67) |
| SDHC | SDHC.4 | TACCTGTAGATAGTAATGTG | UACCUGUAGAUAGUAAUGUG |
| | | (SEQ ID NO: 52) | (SEQ ID NO: 68) |
| DHRS7B | DHRS7B.1 | AATGCTGGGATCAGCTACCG | AAUGCUGGGAUCAGOUACCG |
| | | (SEQ ID NO: 53) | (SEQ ID NO: 69) |
| DHRS7B | DHRS7B.2 | CCCAGAGTCTGTGAGGTCGA | CCCAGAGUCUGUGAGGUCGA |
| | | (SEQ ID NO: 54) | (SEQ ID NO: 70) |
| DHRS7B | DHRS7B.3 | GGTGCTAAACTGGTGCTCTG | GGUGCUAAACUGGUGCUCUG |
| | | (SEQ ID NO: 55) | (SEQ ID NO: 71) |
| DHRS7B | DHRS7B.4 | TGCTGCTGATGGCGACAATG | UGCUGCUGAUGGCGACAAUG |
| | | (SEQ ID NO: 56) | (SEQ ID NO: 72) |

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A composition for treating a subject having Prader Willi Syndrome (PWS) or a PWS-like disorder, the composition comprising an inhibitor of a gene selected from OGDH, LIPT1, SDHC, and DHRS7B.

Clause 2. A composition for activating SNRPN, SPA1, SPA2, or SNORD116, or a combination thereof, the composition comprising an inhibitor of a gene selected from OGDH, LIPT1, SDHC, and DHRS7B.

Clause 3. The composition of clause 1 or 2, wherein the composition reduces expression of the gene selected from OGDH, LIPT1, SDHC, and DHRS7B, or wherein the composition reduces an activity of a protein encoded by the gene selected from OGDH, LIPT1, SDHC, and DHRS7B.

Clause 4. The composition of one of clauses 1-3, wherein the inhibitor comprises a small molecule, a polynucleotide, a polypeptide, or a combination thereof.

Clause 5. The composition of any one of clauses 1-4, wherein the polynucleotide comprises an inhibitory nucleic acid selected from an antisense oligonucleotide, siRNA, RNAi, shRNA, LNA, and PNA.

Clause 6. The composition of clause 5, wherein the inhibitory nucleic acid comprises one or more of a modified internucleoside linkage, a modified sugar moiety, and/or a modified nucleobase.

Clause 7. The composition of any one of clauses 1-4, wherein the inhibitor comprises an antibody.

Clause 8. The composition of any one of clauses 1-4, wherein the inhibitor comprises a DNA Targeting System.

Clause 9. The composition of clause 8, wherein the DNA Targeting System comprises: (a) a zinc finger protein or TALE or DNA binding fusion protein that targets the gene selected from OGDH, LIPT1, SDHC, and DHRS7B; or (b) a CRISPR/Cas9 system that targets the gene selected from OGDH, LIPT1, SDHC, and DHRS7B.

Clause 10. The composition of clause 9, wherein the DNA binding fusion protein comprises a zinc finger protein or TALE, and a polypeptide domain having transcription repression activity and/or nuclease activity.

Clause 11. The composition of clause 10, wherein the polypeptide domain having transcription repression activity comprises KRAB, MECP2, Mad mSIN3 interaction domain (SID), ERF repressor domain (ERD), SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A. Laminin B, and/or CTCF.

Clause 12. The composition of clause 10, wherein the polypeptide domain having nuclease activity comprises FokI.

Clause 13. The composition of clause 9, wherein the CRISPR/Cas9 system comprises: (a) a Cas9 protein or a fusion protein comprising the Cas9 protein; and (b) a gRNA targeting the gene selected from OGDH, LIPT1, SDHC, and DHRS7B, or a portion thereof.

Clause 14. The composition of clause 13, wherein the Cas9 protein is a Streptococcus pyogenes Cas9 protein or a Staphylococcus aureus Cas9 protein.

Clause 15. The composition of clause 14, wherein the Streptococcus pyogenes Cas9 protein comprises the polypeptide sequence of SEQ ID NO: 18, and wherein the Staphylococcus aureus Cas9 protein comprises the polypeptide sequence of SEQ ID NO: 19.

Clause 16. The composition of clause 13, wherein the Cas9 protein is nuclease-deficient dCas9 and comprises the polypeptide sequence of SEQ ID NO: 20 or 21 or is encoded by a polynucleotide sequence comprising SEQ ID NO: 22 or 23.

Clause 17. The composition of any one of clauses 13-16, wherein the fusion protein comprises two heterologous polypeptide domains, wherein the first polypeptide domain comprises the Cas9 protein, and wherein the second polypeptide domain has an activity selected from transcription activation activity, transcription repression activity, transcription release factor activity, histone modification activity, nuclease activity, nucleic acid association activity, methylase activity, and demethylase activity.

Clause 18. The composition of clause 17, wherein the second polypeptide domain has transcription repression activity and/or nuclease activity.

Clause 19. The composition of clause 18, wherein the second polypeptide domain comprises KRAB, MECP2, Mad mSIN3 interaction domain (SID), ERF repressor domain (ERD), SUV39H1, SUV39H2. G9A, ESET/SETBD1, Cir4, Su(var)3-9, Pr-SET7/8, SUV4-20H1, PR-set7, Suv4-20, Set9, EZH2, RIZ1, JMJD2A/JHDM3A. JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, and/or FokI.

Clause 20. The composition of clause 19, wherein the fusion protein comprises dCas9-KRAB.

Clause 21. The composition of any one of clauses 13-20, wherein the gene is OGDH and the gRNA binds and targets a polynucleotide sequence selected from SEQ ID NOs: 41-44, or wherein the gene is LIPT1 and the gRNA binds and targets a polynucleotide sequence selected from SEQ ID NOs: 45-48, or wherein the gene is SDHC and the gRNA binds and targets a polynucleotide sequence selected from SEQ ID NOs: 49-52, or wherein the gene is DHRS7B and the gRNA binds and targets a polynucleotide sequence selected from SEQ ID NOs: 53-56.

Clause 22. A guide RNA (gRNA) that binds and targets a polynucleotide sequence selected from SEQ ID NOs: 41-56, a complement thereof, a variant thereof, or fragment thereof, or that comprises a polynucleotide sequence selected from SEQ ID NOs: 57-72, a complement thereof, a variant thereof, or fragment thereof.

Clause 23. A polynucleotide encoding the composition of any one of clauses 1-22 or at least one component thereof, or the gRNA of clause 22.

Clause 24. A vector comprising the polynucleotide of clause 23.

Clause 25. The vector of clause 24, wherein the vector is a viral vector.

Clause 26. The vector of clause 24, wherein the vector is a retroviral vector, lentiviral vector, adenoviral vector, adeno-associated virus (AAV) vector, synthetic vector, or vector encapsulated within a lipid nanoparticle.

Clause 27. A pharmaceutical composition comprising the composition of any one of clauses 1-21 or at least one component thereof, the gRNA of clause 22, the polynucleotide of clause 23, or the vector of any one of clauses 24-26, or a combination thereof.

Clause 28. A method of treating a subject having PWS or a PWS-like disorder, the method comprising administering to the subject the pharmaceutical composition of clause 27.

Clause 29. The method of clause 28, wherein the subject has a PWS Type 1 large deletion, a PWS Type 2 large deletion, a PWS imprinting center mutation, PWS uniparental disomy, a PWS microdeletion encompassing SNORD116 but not MAGEL2, a PWS or PWS-like atypical deletion encompassing MAGEL2 but not SNORD116, heterozygous Schaaf-Yang syndrome, or MAGEL2 disorder.

Clause 30. The method of any one of clauses 28-29, wherein expression of a gene within the maternal copy of the 15q11-13 locus is increased in the subject.

Clause 31. A method of activating a gene selected from SNRPN, SPA1, SPA2, and SNORD116, or a combination thereof, in a subject in need thereof, the method comprising administering to the subject the pharmaceutical composition of clause 27.

### Sequences

SEQ ID NO: 1
NRG (R = A or G; N can be any nucleotide residue, e.g., any of A, G, C, or T)
SEQ ID NO: 2
NGG (N can be any nucleotide residue, e.g., any of A, G, C, or T)
SEQ ID NO: 3
NAG (N can be any nucleotide residue, e.g., any of A, G, C, or T)
SEQ ID NO: 4
NGGNG (N can be any nucleotide residue, e.g., any of A, G, C, or T)
SEQ ID NO: 5
NNAGAAW (W = A or T; N can be any nucleotide residue, e.g., any of A, G, C, or T)
SEQ ID NO: 6
NAAR (R = A or G; N can be any nucleotide residue, e.g., any of A, G, C, or T)
SEQ ID NO: 7
NNGRR (R = A or G; N can be any nucleotide residue, e.g., any of A, G, C, or T)
SEQ ID NO: 8
NNGRRN (R = A or G; N can be any nucleotide residue, e.g., any of A, G, C, or T)
SEQ ID NO: 9
NNGRRT (R = A or G; N can be any nucleotide residue, e.g., any of A, G, C, or T)
SEQ ID NO: 10
NNGRRV (R = A or G; V = A or C or G; N can be any nucleotide residue, e.g.,
any of A, G, C, or T)
SEQ ID NO: 11
NNNNGATT (N can be any nucleotide residue, e.g., any of A, G, C, or T)
SEQ ID NO: 12
NNNNGNNN (N can be any nucleotide residue, e.g., any of A, G, C, or T)
SEQ ID NO: 13
NGA (N can be any nucleotide residue, e.g., any of A, G, C, or T)
SEQ ID NO: 14
NNNRRT (R = A or G; N can be any nucleotide residue, e.g., any of A, G, C, or T)
SEQ ID NO: 15
ATTCCT
SEQ ID NO: 16
NGAN (N can be any nucleotide residue, e.g., any of A, G, C, or T)
SEQ ID NO: 17
NGNG (N can be any nucleotide residue, e.g., any of A, G, C, or T)
SEQ ID NO: 18
Streptococcus pyogenes Cas9
MDKKYSIGLDIGTNSVGWAVITDEYKVPSKKEKVLGNTDRHSIKKNLIGALLEDSGETAEAT
EVAYHEKYPTIYHLRKKLVDSTDKADLRLIYLALAHMIKERGHFLIEGDLNPDNSDVDKLFI
QLVQTYNQLFEENPINASGVDAKAILSARLSKSRRLENLIAQLPGEKKNGLFGNLIALSLGL
TPNEKSNFDLAEDAKLQLSKDTYDDDLDNLLAQIGDQYADLFLAAKNLSDAILLSDILRVNT
EITKAPLSASMIKRYDEHHQDLTLLKALVROQLPEKYKEIFFDQSKNGYAGYIDGGASQEEF
YKFIKPILEKMDGTEELLVKLNREDLLRKQRTFDNGSIPHQIHLGELHAILRRQEDFYPELK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDELDNEENEDILEDIV
LTLTLFEDREMIEERLKTYAHLFDDKVMKQLKRRRYTGWGRLSRKLINGIRDKQSGKTILDE
LKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETROITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLEVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQ
LGGD
SEQ ID NO: 19
Staphylococcus aureus Cas9 molecule
MKRNYILGLDIGITSVGYGIIDYETRDVIDAGVRLFKEANVENNEGRRSKRGARRLKRRRRH
RIQRVKKLLEDYNLLTDHSELSGINPYEARVKGLSQKLSEEEFSAALLHLAKREGVHNVNEV
EEDTGNELSTKEQISRNSKALEEKYVAELQLERLKKDGEVRGSINREKTSDYVKEAKQLLKV
QKAYHQLDQSFIDTYIDLLETRRTYYEGPGEGSPFGWKDIKEWYEMLMGHCTYFPEELRSVK
YAYNADLYNALNDLNNLVITRDENEKLEYYEKFQIIENVFKQKKKPTLKQIAKEILVNEEDI
KGYRVTSTGKPEFTNLKVYHDIKDITARKEITENAELLDQIAKILTIYQSSEDIQEELTNLN
SELTQEEIEQISNLKGYTGTHNLSLKAINLILDELWHTNDNQIAIFNRLKLVPKKVDLSQQK
EIPTTLVDDFILSPVVKRSFIQSIKVINAIIKKYGLPNDIIIELAREKNSKDAQKMINEMQK
RNRQTNERIEEIIRTTGKENAKYLIEKIKLHDMQEGKCLYSLEAIPLEDLLNNPENYEVDHI
IPRSVSFDNSENNKVLVKQEENSKKGNRTPFQYLSSSDSKISYETFKKHILNLAKGKGRISK
TKKEYLLEERDINRFSVQKDFINRNLVDTRYATRGLMNLLRSYFRVNNLDVKVKSINGGETS
FLRRKWKFKKERNKGYKHHAEDALIIANADFIFKEWKKLDKAKKVMENQMFEEKQAESMPEI
ETEQEYKEIFITPHQIKHIKDFKDYKYSHRVDKKPNRELINDTLYSTRKDDKGNTLIVNNLN
GLYDKDNDKLKKLINKSPEKLLMYHHDPQTYQKLKLIMEQYGDEKNPLYKYYEETGNYLTKY
SKKDNGPVIKKIKYYGNKLNAHLDITDDYPNSRNKVVKLSLKPYRFDVYLDNGVYKEVTVKN
LDVIKKENYYEVNSKCYEEAKKLKKISNQAEFIASFYNNDLIKINGELYRVIGVNNDLLNRI
EVNMIDITYREYLENMNDKRPPRIIKTIASKTQSIKKYSTDILGNLYEVKSKKHPQIIKKG
SEQ ID NO: 20
Streptococcus pyogenes Cas9 (with D10A)
MDKKYSIGLAIGTNSVGWAVITDEYKVPSKKFKVLGNTDRHSIKKNLIGALLEDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNEKSNFDLAEDAKLQLSKDTYDDDLDNLLAQIGDQYADLFLAAKNLSDAILLSDILRVNT
EITKAPLSASMIKRYDEHHQDLTLLKALVROQLPEKYKEIFFDQSKNGYAGYIDGGASQEEF
YKFIKPILEKMDGTEELLVKLNREDLLRKORTFDNGSIPHQIHLGELHAILRRQEDFYPELK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLEKTNRK
VTVKQLKEDYFKKIECFDSVEISGVEDRENASLGTYHDLLKIIKDKDFLDNEENEDILEDIV
LTLTLFEDREMIEERLKTYAHLFDDKVMKQLKRRRYTGWGRLSRKLINGIRDKQSGKTILDE
LKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRGKSD
NVPSEEVVKKMKNYWRQLLNAKLITQRKEDNLTKAERGGLSELDKAGFIKRQLVETRQITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
HYEKLKGSPEDNEQKQLEVEQHKHYLDEIIEQISEFSKRVILADANLDKVLSAYNKHRDKPI
REQAENIIHLETLINLGAPAAFKYFDTTIDRKRYTSTKEVLDATLIHQSITGLYETRIDLSQ
LGGD
SEQ ID NO: 21
Streptococcus pyogenes Cas9 (with D10A, H849A)
MDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNEKSNFDLAEDAKLQLSKDTYDDDLDNLLAQIGDQYADLFLAAKNLSDAILLSDILRVNT
EITKAPLSASMIKRYDEHHODLTLLKALVRQQLPEKYKEIFFDQSKNGYAGYIDGGASQEEE
YKFIKPILEKMDGTEELLVKLNREDLLRKQRTFDNGSIPHQIHLGELHAILRRQEDFYPELK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRENASLGTYHDLLKIIKDKDFLDNEENEDILEDIV
LTLTLFEDREMIEERLKTYAHLFDDKVMKQLKRRRYTGWGRLSRKLINGIRDKQSGKTILDE
LKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVLTRSDKNRGKSD
NVPSEEVVKKMKNYWRQLLNAKLITQRKEDNITKAERGGLSELDKAGFIKRQLVETRQITKH
VAQILDSRMNTKYDENDKLIREVKVITLKSKLVSDFRKDFQFYKVREINNYHHAHDAYINAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLEVEQHKHYLDEIIEQISEFSKRVILADANLDKVLSAYNKHRDKPI
REQAENIIHLETLTNLGAPAAFKYFDTTIDRKRYTSTKEVLDATLIHQSITGLYETRIDLSQ
LGGD
SEQ ID NO: 22
Polynucleotide sequence of D10A mutant of S. aureus Cas9
atgaaaagga actacattct ggggctggcc atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tcgatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 23
Polynucleotide sequence of N580A mutant of S. aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataad
gtcaatgagg tggaagagga caccggcaac gagctgtcta caaaggaaca gatctcacgd
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagaggcc
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 24
codon optimized polynucleotide encoding S. pyogenes Cas9
atggataaaa agtacagcat cgggctggac atcggtacaa actcagtggg gtgggccgtg
attacggacg agtacaaggt accctccaaa aaatttaaag tgctgggtaa cacggacaga
cactctataa agaaaaatct tattggagcc ttgctgttcg actcaggcga gacagccgaa
gccacaaggt tgaagcggac cgccaggagg cggtatacca ggagaaagaa ccgcatatgc
tacctgcaag aaatcttcag taacgagatg gcaaaggttg acgatagctt tttccatcgc
ctggaagaat cctttcttgt tgaggaagac aagaagcacg aacggcaccc catctttggc
aatattgtcg acgaagtggc atatcacgaa aagtacccga ctatctacca cctcaggaag
aagctggtgg actctaccga taaggcggac ctcagactta tttatttggc actcgcccac
atgattaaat ttagaggaca tttcttgatc gagggcgacc tgaacccgga caacagtgac
gtcgataagc tgttcatcca acttgtgcag acctacaatc aactgttcga agaaaaccct
ataaatgctt caggagtcga cgctaaagca atcctgtccg cgcgcctctc aaaatctaga
agacttgaga atctgattgc tcagttgccc ggggaaaaga aaaatggatt gtttggcaac
ctgatcgccc tcagtctcgg actgacccca aatttcaaaa gtaacttcga cctggccgaa
gacgctaagc tccagctgtc caaggacaca tacgatgacg acctcgacaa tctgctggcc
cagattgggg atcagtacgc cgatctcttt ttggcagcaa agaacctgtc cgacgccatc
ctgttgagcg atatcttgag agtgaacacc gaaattacta aagcacccct tagcgcatct
atgatcaagc ggtacgacga gcatcatcag gatctgaccc tgctgaaggc tcttgtgagg
caacagctcc ccgaaaaata caaggaaatc ttctttgacc agagcaaaaa cggctacgct
ggctatatag atggtggggc cagtcaggag gaattctata aattcatcaa gcccattctc
gagaaaatgg acggcacaga ggagttgctg gtcaaactta acagggagga cctgctgcgg
aagcagcgga cctttgacaa cgggtctatc ccccaccaga ttcatctggg cgaactgcac
gcaatcctga ggaggcagga ggatttttat ccttttctta aagataaccg cgagaaaata
gaaaagattc ttacattcag gatcccgtac tacgtgggac ctctcgcccg gggcaattca
cggtttgcct ggatgacaag gaagtcagag gagactatta caccttggaa cttcgaagaa
gtggtggaca agggtgcatc tgcccagtct ttcatcgagc ggatgacaaa ttttgacaag
aacctcccta atgagaaggt gctgcccaaa cattctctgc tctacgagta ctttaccgtc
tacaatgaac tgactaaagt caagtacgtc accgagggaa tgaggaagcc ggcattcctt
agtggagaac agaagaaggc gattgtagac ctgttgttca agaccaacag gaaggtgact
gtgaagcaac ttaaagaaga ctactttaag aagatcgaat gttttgacag tgtggaaatt
tcaggggttg aagaccgctt caatgcgtca ttggggactt accatgatct tctcaagatc
ataaaggaca aagacttcct ggacaacgaa gaaaatgagg atattctcga agacatcgtc
ctcaccctga ccctgttcga agacagggaa atgatagaag agcgcttgaa aacctatgcc
cacctcttcg acgataaagt tatgaagcag ctgaagcgca ggagatacac aggatgggga
agattgtcaa ggaagctgat caatggaatt agggataaac agagtggcaa gaccatactg
gatttcctca aatctgatgg cttcgccaat aggaacttca tgcaactgat tcacgatgac
tctcttacct tcaaggagga cattcaaaag gctcaggtga gcgggcaggg agactccctt
catgaacaca tcgcgaattt ggcaggttcc cccgctatta aaaagggcat ccttcaaact
gtcaaggtgg tcgatgaatt ggtcaaggta atgggcagac ataagccaga aaatattgtg
atcgagatgg cccgcgaaaa ccagaccaca cagaagggcc agaaaaatag tagagagcgg
atgaagagga tcgaggaggg catcaaagag ctgggatctc agattctcaa agaacacccc
gtagaaaaca cacagctgca gaacgaaaaa ttgtacttgt actatctgca gaacggcaga
gacatgtacg tcgaccaaga acttgatatt aatagactgt ccgactatga cctagaccat
atcgtgcccc agtccttcct gaaggacgac tccattgata acaaagtctt gacaagaagc
gacaagaaca ggggtaaaag tgataatgtg cctagcgagg aggtggtgaa aaaaatgaag
aactactggc gacagctgct taatgcaaag ctcattacac aacggaagtt cgataatctg
acgaaagcag agagaggtgg cttgtctgag ttggacaagg cagggtttat taagcggcag
ctggtggaaa ctaggcagat cacaaagcac gtggcgcaga ttttggacag ccggatgaac
acaaaatacg acgaaaatga taaactgata cgagaggtca aagttatcac gctgaaaagc
aagctggtgt ccgattttcg gaaagacttc cagttctaca aagttcgcga gattaataac
taccatcatg ctcacgatgc gtacctgaac gctgttgtcg ggaccgcctt gataaagaag
tacccaaagc tggaatccga gttcgtatac ggggattaca aagtgtacga tgtgaggaaa
atgatagcca agtccgagca ggagattgga aaggccacag ctaagtactt cttttattct
aacatcatga atttttttaa gacggaaatt accctggcca acggagagat cagaaagcgg
ccccttatag agacaaatgg tgaaacaggt gaaatcgtct gggataaggg cagggatttc
gctactgtga ggaaggtgct gagtatgcca caggtaaata tcgtgaaaaa aaccgaagta
cagaccggag gattttccaa ggaaagcatt ttgcctaaaa gaaactcaga caagctcatc
gcccgcaaga aagattggga ggaaagcatt ttgcctaaaa gaaactcaga caagctcatc
tattctgtgc tggtggtagc ccctaagaaa tacgggggat ttgactcacc caccgtagcc
aaggaactct tgggaatcac taaggtggaa aaaggaaagt ctaagaagct gaagtccgtg
ttcctggagg ctaagggtta tatcatggaa agatcatcct ttgaaaagaa ccctatcgat
tactctctct tcgagctgga aaatggcagg aagagaatgt tggccagcgc cggagagctg
caaaagggaa acgagcttgc tctgccctcc aaatatgtta attttctcta tctcgcttcc
cactatgaaa agctgaaagg gtctcccgaa gataacgagc agaagcagct gttcgtcgaa
cagcacaagc actatctgga tgaaataatc gaacaaataa gcgagttcag caaaagggtt
atcctggcgg atgctaattt ggacaaagta ctgtctgctt ataacaagca ccgggataag
cctattaggg aacaagccga gaatataatt cacctcttta cactcacgaa tctcggagcc
cccgccgcct tcaaatactt tgatacgact atcgaccgga aacggtatac cagtaccaaa
gaggtcctcg atgccaccct catccaccag tcaattactg gcctgtacga aacacggatc
gacctctctc aactgggcgg cgactag
SEQ ID NO: 25
codon optimized nucleic acid sequences encoding S. aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
tccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tcgagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 26
codon optimized nucleic acid sequences encoding S. aureus Cas9
atgaagcgga actacatcct gggcctggac atcggcatca ccagcgtggg ctacggcatc
atcgactacg agacacggga cgtgatcgat gccggcgtgc ggctgttcaa agaggccaac
gtggaaaaca acgagggcag gcggagcaag agaggcgcca gaaggctgaa gcggcggagg
cggcatagaa tccagagagt gaagaagctg ctgttcgact acaacctgct gaccgaccac
agcgagctga gcggcatcaa cccctacgag gccagagtga agggcctgag ccagaagctg
agcgaggaag agttctctgc cgccctgctg cacctggcca agagaagagg cgtgcacaac
gtgaacgagg tcgaagagga caccggcaac gagctgtcca ccaaagagca gatcagccgg
aacagcaagg ccctggaaga gaaatacgtg gccgaactgc agctggaacg gctgaagaaa
gacggcgaag tgcggggcag catcaacaga ttcaagacca gcgactacgt gaaagaagcc
aaacagctgc tgaaggtgca gaaggcctac caccagctgg accagagctt catcgacacc
tacatcgacc tgctggaaac ccggcggacc tactatgagg gacctggcga gggcagcccc
ttcggctgga aggacatcaa agaatggtac gagatgctga tgggccactg cacctacttc
cccgaggaac tgcggagcgt gaagtacgcc tacaacgccg acctgtacaa cgccctgaac
gacctgaaca atctcgtgat caccagggac gagaacgaga agctggaata ttacgagaag
ttccagatca tcgagaacgt gttcaagcag aagaagaagc ccaccctgaa gcagatcgcc
aaagaaatcc tcgtgaacga agaggatatt aagggctaca gagtgaccag caccggcaag
cccgagttca ccaacctgaa ggtgtaccac gacatcaagg acattaccgc ccggaaagag
attattgaga acgccgagct gctggatcag attgccaaga tcctgaccat ctaccagagc
agcgaggaca tccaggaaga actgaccaat ctgaactccg agctgaccca ggaagagatc
gagcagatct ctaatctgaa gggctatacc ggcacccaca acctgagcct gaaggccatc
aacctgatcc tggacgagct gtggcacacc aacgacaacc agatcgctat cttcaaccgg
ctgaagctgg tgcccaagaa ggtggacctg tcccagcaga aagagatccc caccaccctg
gtggacgact tcatcctgag ccccgtcgtg aagagaagct tcatccagag catcaaagtg
atcaacgcca tcatcaagaa gtacggcctg cccaacgaca tcattatcga gctggcccgc
gagaagaact ccaaggacgc ccagaaaatg atcaacgaga tgcagaagcg gaaccggcag
accaacgagc ggatcgagga aatcatccgg accaccggca aagagaacgc caagtacctg
atcgagaaga tcaagctgca cgacatgcag gaaggcaagt gcctgtacag cctggaagcc
atccctctgg aagatctgct gaacaacccc ttcaactatg aggtggacca catcatcccc
agaagcgtgt ccttcgacaa cagcttcaac aacaaggtgc tcgtgaagca ggaagaaaac
agcaagaagg gcaaccggac cccattccag tacctgagca gcagcgacag caagatcagc
tacgaaacct tcaagaagca catcctgaat ctggccaagg gcaagggcag aatcagcaag
accaagaaag agtatctgct ggaagaacgg gacatcaaca ggttctccgt gcagaaagac
ttcatcaacc ggaacctggt ggataccaga tacgccacca gaggcctgat gaacctgctg
cggagctact tcagagtgaa caacctggac gtgaaagtga agtccatcaa tggcggcttc
accagctttc tgcggcggaa gtggaagttt aagaaagagc ggaacaaggg gtacaagcac
cacgccgagg acgccctgat cattgccaac gccgatttca tcttcaaaga gtggaagaaa
ctggacaagg ccaaaaaagt gatggaaaac cagatgttcg aggaaaagca ggccgagagc
atgcccgaga tcgaaaccga gcaggagtac aaagagatct tcatcacccc ccaccagatc
aagcacatta aggacttcaa ggactacaag tacagccacc gggtggacaa gaagcctaat
agagagctga ttaacgacac cctgtactcc acccggaagg acgacaaggg caacaccctg
atcgtgaaca atctgaacgg cctgtacgac aaggacaatg acaagctgaa aaagctgatc
aacaagagcc ccgaaaagct gctgatgtac caccacgacc cccagaccta ccagaaactg
aagctgatta tcgaacagta cggcgacgag aagaatcccc tgtacaagta ctacgaggaa
accgggaact acctgaccaa gtactccaaa aaggacaacg gccccgtgat caagaagatt
aagtattacg gcaacaaact gaacgcccat ctggacatca ccgacgacta ccccaacagc
agaaacaagg tcgtgaagct gtccctgaag ccctacagat tcgacgtgta cctggacaat
ggcgtgtaca agttcgtgac cgtgaagaat ctggatgtga tcaaaaaaga aaactactac
gaagtgaata gcaagtgcta tgaggaagct aagaagctga agaagatcag caaccaggcc
gagtttatcg cctccttcta caacaacgat ctgatcaaga tcaacggcga gctgtataga
gtgatcggcg tgaacaacga cctgctgaac cggatcgaag tgaacatgat cgacatcacc
taccgcgagt acctggaaaa catgaacgac aagaggcccc ccaggatcat taagacaatc
gcctccaaga cccagagcat taagaagtac agcacagaca ttctgggcaa cctgtatgaa
gtgaaatcta agaagcaccc tcagatcatc aaaaagggc
SEQ ID NO: 27
codon optimized nucleic acid sequence encoding S. aureus Cas9
atgaagcgca actacatcct cggactggac atcggcatta cctccgtggg atacggcatc
atcgattacg aaactaggga tgtgatcgac gctggagtca ggctgttcaa agaggcgaac
gtggagaaca acgaggggcg gcgctcaaag aggggggccc gccggctgaa gcgccgccgc
agacatagaa tccagcgcgt gaagaagctg ctgttcgact acaaccttct gaccgaccac
tccgaacttt ccggcatcaa cccatatgag gctagagtga agggattgtc ccaaaagctg
tccgaggaag agttctccgc cgcgttgctc cacctcgcca agcgcagggg agtgcacaat
gtgaacgaag tcgaagaaga taccggaaac gagctgtcca ccaaggagca gatcagccgg
aactccaagg ccctggaaga gaaatacgtg gcggaactgc aactggagcg gctgaagaaa
gacggagaag tgcgcggctc gatcaaccgc ttcaagacct cggactacgt gaaggaggcc
aagcagctcc tgaaagtgca aaaggcctat caccaacttg accagtcctt tatcgatacc
tacatcgatc tgctcgagac tcggcggact tactacgagg gtccagggga gggctcccca
tttggttgga aggatattaa ggagtggtac gaaatgctga tgggacactg cacatacttc
cctgaggagc tgcggagcgt gaaatacgca tacaacgcag acctgtacaa cgcgctgaac
gacctgaaca atctcgtgat cacccgggac gagaacgaaa agctcgagta ttacgaaaag
ttccagatta ttgagaacgt gttcaaacag aagaagaagc cgacactgaa gcagattgcc
aaggaaatcc tcgtgaacga agaggacatc aagggctatc gagtgacctc aacgggaaag
ccggagttca ccaatctgaa ggtctaccac gacatcaaag acattaccgc ccggaaggag
atcattgaga acgcggagct gttggaccag attgcgaaga ttctgaccat ctaccaatcc
tccgaggata ttcaggaaga actcaccaac ctcaacagcg aactgaccca ggaggagata
gagcaaatct ccaacctgaa gggctacacc ggaactcata acctgagcct gaaggccatc
aacttgatcc tggacgagct gtggcacacc aacgataacc agatcgctat tttcaatcgg
ctgaagctgg tccccaagaa agtggacctc tcacaacaaa aggagatccc tactaccctt
gtggacgatt tcattctgtc ccccgtggtc aagagaagct tcatacagtc aatcaaagtg
atcaatgcca ttatcaagaa atacggtctg cccaacgaca ttatcattga gctcgcccgc
gagaagaact cgaaggacgc ccagaagatg attaacgaaa tgcagaagag gaaccgacag
actaacgaac ggatcgaaga aatcatccgg accaccggga aggaaaacgc gaagtacctg
atcgaaaaga tcaagctcca tgacatgcag gaaggaaagt gtctgtactc gctggaggcc
attccgctgg aggacttgct gaacaaccct tttaactacg aagtggatca tatcattccg
aggagcgtgt cattcgacaa ttccttcaac aacaaggtcc tcgtgaagca ggaggaaaac
tcgaagaagg gaaaccgcac gccgttccag tacctgagca gcagcgactc caagatttcc
tacgaaacct tcaagaagca catcctcaac ctggcaaagg ggaagggtcg catctccaag
accaagaagg aatatctgct ggaagaaaga gacatcaaca gattctccgt gcaaaaggac
ttcatcaacc gcaacctcgt ggatactaga tacgctactc ggggtctgat gaacctcctg
agaagctact ttagagtgaa caatctggac gtgaaggtca agtcgattaa cggaggtttc
acctccttcc tgcggcgcaa gtggaagttc aagaaggaac ggaacaaggg ctacaagcac
cacgccgagg acgccctgat cattgccaac gccgacttca tcttcaaaga atggaagaaa
cttgacaagg ctaagaaggt catggaaaac cagatgttcg aagaaaagca ggccgagtct
atgcctgaaa tcgagactga acaggagtac aaggaaatct ttattacgcc acaccagatc
aaacacatca aggatttcaa ggattacaag tactcacatc gcgtggacaa aaagccgaac
agggaactga tcaacgacac cctctactcc acccggaagg atgacaaagg gaataccctc
atcgtcaaca accttaacgg cctgtacgac aaggacaacg ataagctgaa gaagctcatt
aacaagtcgc ccgaaaagtt gctgatgtac caccacgacc ctcagactta ccagaagctc
aagctgatca tggagcagta tggggacgag aaaaacccgt tgtacaagta ctacgaagaa
actgggaatt atctgactaa gtactccaag aaagataacg gccccgtgat taagaagatt
aagtactacg gcaacaagct gaacgcccat ctggacatca ccgatgacta ccctaattcc
cgcaacaagg tcgtcaagct gagcctcaag ccctaccggt ttgatgtgta ccttgacaat
ggagtgtaca agttcgtgac tgtgaagaac cttgacgtga tcaagaagga gaactactac
gaagtcaact ccaagtgcta cgaggaagca aagaagttga agaagatctc gaaccaggcc
gagttcattg cctccttcta taacaacgac ctgattaaga tcaacggcga actgtaccgc
gtcattggcg tgaacaacga tctcctgaac cgcatcgaag tgaacatgat cgacatcact
taccgggaat acctggagaa tatgaacgac aagcgcccgc cccggatcat taagactatc
gcctcaaaga cccagtcgat caagaagtac agcaccgaca tcctgggcaa cctgtacgag
gtcaaatcga agaagcaccc ccagatcate aagaaggga
SEQ ID NO: 28
codon optimized nucleic acid sequence encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccagagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaggcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
SEQ ID NO: 29
codon optimized nucleic acid sequence encoding S. aureus Cas9
accggtgcca ccatgtaccc atacgatgtt ccagattacg cttcgccgaa gaaaaagcgc
aaggtcgaag cgtccatgaa aaggaactac attctggggc tggacatcgg gattacaagc
gtggggtatg ggattattga ctatgaaaca agggacgtga tcgacgcagg cgtcagactg
ttcaaggagg ccaacgtgga aaacaatgag ggacggagaa gcaagagggg agccaggcgc
ctgaaacgac ggagaaggca cagaatccag agggtgaaga aactgctgtt cgattacaac
ctgctgaccg accattctga gctgagtgga attaatcctt atgaagccag ggtgaaaggc
ctgagtcaga agctgtcaga ggaagagttt tccgcagctc tgctgcacct ggctaagcgc
cgaggagtgc ataacgtcaa tgaggtggaa gaggacaccg gcaacgagct gtctacaaag
gaacagatct cacgcaatag caaagctctg gaagagaagt atgtcgcaga gctgcagctg
gaacggctga agaaagatgg cgaggtgaga gggtcaatta ataggttcaa gacaagcgac
tacgtcaaag aagccaagca gctgctgaaa gtgcagaagg cttaccacca gctggatcag
agcttcatcg atacttatat cgacctgctg gagactcgga gaacctacta tgagggacca
ggagaaggga gccccttcgg atggaaagac atcaaggaat ggtacgagat gctgatggga
cattgcacct attttccaga agagctgaga agcgtcaagt acgcttataa cgcagatct
tacaacgccc tgaatgacct gaacaacctg gtcatcacca gggatgaaaa cgagaaactg
gaatactatg agaagttcca gatcatcgaa aacgtgttta agcagaagaa aaagcctaca
ctgaaacaga ttgctaagga gatcctggtc aacgaagagg acatcaaggg ctaccgggtg
acaagcactg gaaaaccaga gttcaccaat ctgaaagtgt atcacgatat taaggacatc
acagcacgga aagaaatcat tgagaacgcc gaactgctgg atcagattgc taagatcctg
actatctacc agagctccga ggacatccag gaagagctga ctaacctgaa cagcgagctg
acccaggaag agatcgaaca gattagtaat ctgaaggggt acaccggaac acacaacctg
tccctgaaag ctatcaatct gattctggat gagctgtggc atacaaacga caatcagatt
gcaatcttta accggctgaa gctggtccca aaaaaggtgg acctgagtca gcagaaagag
atcccaacca cactggtgga cgatttcatt ctgtcacccg tggtcaagcg gagcttcatc
cagagcatca aagtgatcaa cgccatcatc aagaagtacg gcctgcccaa tgatatcatt
atcgagctgg ctagggagaa gaacagcaag gacgcacaga agatgatcaa tgagatgcag
aaacgaaacc ggcagaccaa tgaacgcatt gaagagatta tccgaactac ccggaaagag
aacgcaaagt acctgattga aaaaatcaag ctgcacgata tgcaggaggg aaagtgtctg
tattctctgg aggccatccc cctggaggac ctgctgaaca atccattcaa ctacgaggtc
gatcatatta tccccagaag cgtgtccttc gacaattcct ttaacaacaa ggtgctggtc
aagcaggaag agaactctaa aaagggcaat aggactcctt tccagtacct gtctagttca
gattccaaga tctcttacga aacctttaaa aagcacattc tgaatctggc caaaggaaag
ggccgcatca gcaagaccaa aaaggagtac ctgctggaag agcgggacat caacagattc
tccgtccaga aggattttat taaccggaat ctggtggaca caagatacgc tactcgcggc
ctgatgaatc tgctgcgatc ctatttccgg gtgaacaatc tggatgtgaa agtcaagtcc
atcaacggcg ggttcacatc ttttctgagg cgcaaatgga agtttaaaaa ggagcgcaac
aaagggtaca agcaccatgc cgaagatgct ctgattatcg caaatgccga cttcatcttt
aaggagtgga aaaagctgga caaagccaag aaagtgatgg agaaccagat gttcgaagag
aagcaggccg aatctatgcc cgaaatcgag acagaacagg agtacaagga gattttcatc
actcctcacc agatcaagca tatcaaggat ttcaaggact acaagtactc tcaccgggtg
gataaaaagc ccaacagaga gctgatcaat gacaccctgt atagtacaag aaaagacgat
aaggggaata ccctgattgt gaacaatctg aacggactgt acgacaaaga taatgacaag
ctgaaaaagc tgatcaacaa aagtcccgag aagctgctga tgtaccacca tgatcctcag
acatatcaga aactgaagct gattatggag cagtacggcg acgagaagaa cccactgtat
aagtactatg aagagactgg gaactacctg accaagtata gcaaaaagga taatggcccc
gtgatcaaga agatcaagta ctatgggaac aagctgaatg cccatctgga catcacagac
gattacccta acagtcgcaa caaggtggtc aagctgtcac tgaagccata cagattcgat
gtctatctgg acaacggcgt gtataaattt gtgactgtca agaatctgga tgtcatcaaa
aaggagaact actatgaagt gaatagcaag tcctacgaag aggctaaaaa gctgaaaaag
attagcaacc aggcagagtt catcgcctcc ttttacaaca acgacctgat taagatcaat
ggcgaactgt atagggtcat cggggtgaac aatgatctgc tgaaccgcat tgaagtgaat
atgattgaca tcacttaccg agagtatctg gaaaacatga atgataagcg cccccctcga
attatcaaaa caattgcctc taagactcag agtatcaaaa agtactcaac cgacattctg
ggaaacctgt atgaggtgaa gagcaaaaag caccctcaga ttatcaaaaa gggctaagaa
ttc
SEQ ID NO: 30
codon optimized nucleic acid sequences encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggggccacgaaaaaggccggccaggcaaaaaagaaaaag
SEQ ID NO: 31
codon optimized nucleic acid sequences encoding S. aureus Cas9
aagcggaactacatcctgggcctggacatcggcatcaccagcgtgggctacggcatcatcgactacga
gacacgggacgtgatcgatgccggcgtgcggctgttcaaagaggccaacgtggaaaacaacgagggca
ggcggagcaagagaggcgccagaaggctgaagcggcggaggcggcatagaatccagagagtgaagaag
ctgctgttcgactacaacctgctgaccgaccacagcgagctgagcggcatcaacccctacgaggccag
agtgaagggcctgagccagaagctgagcgaggaagagttctctgccgccctgctgcacctggccaaga
gaagaggcgtgcacaacgtgaacgaggtggaagaggacaccggcaacgagctgtccaccaaagagcag
atcagccggaacagcaaggccctggaagagaaatacgtggccgaactgcagctggaacggctgaagaa
agacggcgaagtgcggggcagcatcaacagattcaagaccagcgactacgtgaaagaagccaaacagc
tgctgaaggtgcagaaggcctaccaccagctggaccagagcttcatcgacacctacatcgacctgctg
gaaacccggcggacctactatgagggacctggcgagggcagccccttcggctggaaggacatcaaaga
atggtacgagatgctgatgggccactgcacctacttccccgaggaactgcggagcgtgaagtacgcct
acaacgccgacctgtacaacgccctgaacgacctgaacaatctcgtgatcaccagggacgagaacgag
aagctggaatattacgagaagttccagatcatcgagaacgtgttcaagcagaagaagaagcccaccct
gaagcagatcgccaaagaaatcctcgtgaacgaagaggatattaagggctacagagtgaccagcaccg
gcaagcccgagttcaccaacctgaaggtgtaccacgacatcaaggacattaccgcccggaaagagatt
attgagaacgccgagctgctggatcagattgccaagatcctgaccatctaccagagcagcgaggacat
ccaggaagaactgaccaatctgaactccgagctgacccaggaagagatcgagcagatctctaatctga
agggctataccggcacccacaacctgagcctgaaggccatcaacctgatcctggacgagctgtggcac
accaacgacaaccagatcgctatcttcaaccggctgaagctggtgcccaagaaggtggacctgtccca
gcagaaagagatccccaccaccctggtggacgacttcatcctgagccccgtcgtgaagagaagcttca
tccagagcatcaaagtgatcaacgccatcatcaagaagtacggcctgcccaacgacatcattatcgag
ctggcccgcgagaagaactccaaggacgcccagaaaatgatcaacgagatgcagaagcggaaccggca
gaccaacgagcggatcgaggaaatcatccggaccaccggcaaagagaacgccaagtacctgatcgaga
agatcaagctgcacgacatgcaggaaggcaagtgcctgtacagcctggaagccatccctctggaagat
ctgctgaacaaccccttcaactatgaggtggaccacatcatccccagaagcgtgtccttcgacaacag
cttcaacaacaaggtgctcgtgaagcaggaagaaaacagcaagaagggcaaccggaccccattccagt
acctgagcagcagcgacagcaagatcagctacgaaaccttcaagaagcacatcctgaatctggccaag
ggcaagggcagaatcagcaagaccaagaaagagtatctgctggaagaacgggacatcaacaggttctc
cgtgcagaaagacttcatcaaccggaacctggtggataccagatacgccaccagaggcctgatgaacc
tgctgcggagctacttcagagtgaacaacctggacgtgaaagtgaagtccatcaatggcggcttcacc
agctttctgcggcggaagtggaagtttaagaaagagcggaacaaggggtacaagcaccacgccgagga
cgccctgatcattgccaacgccgatttcatcttcaaagagtggaagaaactggacaaggccaaaaaag
tgatggaaaaccagatgttcgaggaaaagcaggccgagagcatgcccgagatcgaaaccgagcaggag
tacaaagagatcttcatcaccccccaccagatcaagcacattaaggacttcaaggactacaagtacag
ccaccgggtggacaagaagcctaatagagagctgattaacgacaccctgtactccacccggaaggacg
acaagggcaacaccctgatcgtgaacaatctgaacggcctgtacgacaaggacaatgacaagctgaaa
aagctgatcaacaagagccccgaaaagctgctgatgtaccaccacgacccccagacctaccagaaact
gaagctgattatggaacagtacggcgacgagaagaatcccctgtacaagtactacgaggaaaccggga
actacctgaccaagtactccaaaaaggacaacggccccgtgatcaagaagattaagtattacggcaac
aaactgaacgcccatctggacatcaccgacgactaccccaacagcagaaacaaggtcgtgaagctgtc
tggatgtgatcaaaaaagaaaactactacgaagtgaatagcaagtgctatgaggaagctaagaagctg
aagaagatcagcaaccaggccgagtttatcgcctccttctacaacaacgatctgatcaagatcaacgg
cgagctgtatagagtgatcggcgtgaacaacgacctgctgaaccggatcgaagtgaacatgatcgaca
tcacctaccgcgagtacctggaaaacatgaacgacaagaggccccccaggatcattaagacaatcgcc
tccaagacccagagcattaagaagtacagcacagacattctgggcaacctgtatgaagtgaaatctaa
gaagcaccctcagatcatcaaaaagggc
SEQ ID NO: 32
Vector (pDO242) encoding codon optimized nucleic acid sequence
encoding S. aureus Cas9
ctaaattgtaagcgttaatattttgttaaaattcgcgttaaatttttgttaaatcagctcatttttta
accaataggccgaaatcggcaaaatcccttataaatcaaaagaatagaccgagatagggttgagtgtt
gttccagtttggaacaagagtccactattaaagaacgtggactccaacgtcaaagggcgaaaaaccgt
ctatcagggcgatggcccactacgtgaaccatcaccctaatcaagttttttggggtcgaggtgccgta
aagcactaaatcggaaccctaaagggagcccccgatttagagcttgacggggaaagccggcgaacgtg
gcgagaaaggaagggaagaaagcgaaaggagcgggcgctagggcgctggcaagtgtagcggtcacgct
gcgcgtaaccaccacacccgccgcgcttaatgcgccgctacagggcgcgtcccattcgccattcaggc
tgcgcaactgttgggaagggcgatcggtgcgggcctcttcgctattacgccagctggcgaaaggggga
tgtgctgcaaggcgattaagttgggtaacgccagggttttcccagtcacgacgttgtaaaacgacggc
cgttgacattgattattgactagttattaatagtaatcaattacggggtcattagttcatagcccata
tatggagttccgcgttacataacttacggtaaatggcccgcctggctgaccgcccaacgacccccgcc
cattgacgtcaataatgacgtatgttcccatagtaacgccaatagggactttccattgacgtcaatgg
gtggagtatttacggtaaactgcccacttggcagtacatcaagtgtatcatatgccaagtacgccccc
tattgacgtcaatgacggtaaatggcccgcctggcattatgcccagtacatgaccttatgggactttc
ctacttggcagtacatctacgtattagtcatcgctattaccatggtgatgcggttttggcagtacatc
aatgggcgtggatagcggtttgactcacggggatttccaagtctccaccccattgacgtcaatgggag
tttgttttggcaccaaaatcaacgggactttccaaaatgtcgtaacaactccgccccattgacgcaaa
tgggcggtaggcgtgtacggtgggaggtctatataagcagagctctctggctaactaccggtgccacc
ATGAAAAGGAACTACATTCIGGGGCTGGACATCGGGATTACAAGCGTGGGGTATGGGATTATTGACTA
TGAAACAAGGGACGTGATCGACGCAGGCGTCAGACTGTTCAAGGAGGCCAACGTGGAAAACAATGAGG
GACGGAGAAGCAAGAGGGGAGCCAGGCGCCTGAAACGACGGAGAAGGCACAGAATCCAGAGGGTGAAG
AAACTGCTGTTCGATTACAACCTGCTGACCGACCATTCTGAGCTGAGTGGAATTAATCCTTATGAAGC
CAGGGTGAAAGGCCTGAGTCAGAAGCTGTCAGAGGAAGAGTTTTCCGCAGCTCTGCTGCACCTGGCTA
AGCGCCGAGGAGTGCATAACGTCAATGAGGTGGAAGAGGACACCGGCAACGAGCTGTCTACAAAGGAA
CAGATCTCACGCAATAGCAAAGOTCTGGAAGAGAAGTATGTCGCAGAGCTGCAGCTGGAACGGCTGAA
GAAAGATGGCGAGGTGAGAGGGTCAATTAATAGGTTCAAGACAAGCGACTACGTCAAAGAAGCCAAGC
AGCTGCTGAAAGTGCAGAAGGCTTACCACCAGCTGGATCAGAGCTTCATCGATACTTATATCGACCTG
CTGGAGACTCGGAGAACCTACTATGAGGGACCAGGAGAAGGGAGCCCCTTCGGATGGAAAGACATCAA
GGAATGGTACGAGATGCTGATGGGACATTGCACCTATTTTCCAGAAGAGCTGAGAAGCGTCAAGTACG
CTTATAACGCAGATCTGTACAACGCCCTGAATGACCTGAACAACCTGGTCATCACCAGGGATGAAAAC
GAGAAACTGGAATACTATGAGAAGTTCCAGATCATCGAAAACGTGTTTAAGCAGAAGAAAAAGCCTAC
ACTGAAACAGATTGCTAAGGAGATCCTGGTCAACGAAGAGGACATCAAGGGCTACCGGGTGACAAGCA
CTGGAAAACCAGAGTTCACCAATCTGAAAGTGTATCACGATATTAAGGACATCACAGCACGGAAAGAA
ATCATTGAGAACGCCGAACTGCTGGATCAGATTGCTAAGATCCTGACTATCTACCAGAGCTCCGAGGA
CATCCAGGAAGAGCTGACTAACCTGAACAGCGAGCTGACCCAGGAAGAGATCGAACAGATTAGTAATC
TGAAGGGGTACACCGGAACACACAACCTGTCCCTGAAAGCTATCAATCTGATTCTGGATGAGCTGTGG
CATACAAACGACAATCAGATTGCAATCTTTAACCGGCTGAAGCTGGTCCCAAAAAAGGTGGACCTGAG
TCAGCAGAAAGAGATCCCAACCACACTGGTGGACGATTTCATTCTGTCACCCGTGGTCAAGCGGAGCT
TCATCCAGAGCATCAAAGTGATCAACGCCATCATCAAGAAGTACGGCCTGCCCAATGATATCATTATC
GAGCTGGCTAGGGAGAAGAACAGCAAGGACGCACAGAAGATGATCAATGAGATGCAGAAACGAAACCG
GCAGACCAATGAACGCATTGAAGAGATTATCCGAACTACCGGGAAAGAGAACGCAAAGTACCTGATTG
AAAAAATCAAGCTGCACGATATGCAGGAGGGAAAGTGTCTGTATTCTCTGGAGGCCATCCCCCTGGAG
GACCTGCTGAACAATCCATTCAACTACGAGGTCGATCATATTATCCCCAGAAGCGTGTCCTTCGACAA
TTCCTTTAACAACAAGGTGCTGGTCAAGCAGGAAGAGAACTCTAAAAAGGGCAATAGGACTCCTTTCC
AGTACCTGTCTAGTTCAGATTCCAAGATCTCTTACGAAACCTTTAAAAAGCACATTCTGAATCTGGCC
AAAGGAAAGGGCCGCATCAGCAAGACCAAAAAGGAGTACCTGCTGGAAGAGCGGGACATCAACAGATT
CTCCGTCCAGAAGGATTTTATTAACCGGAATCTGGTGGACACAAGATACGCTACTCGCGGCCTGATGA
ATCTGCTGCGATCCTATTTCCGGGTGAACAATCTGGATGTGAAAGTCAAGTCCATCAACGGCGGGTTC
ACATCTTTTCTGAGGCGCAAATGGAAGTTTAAAAAGGAGCGCAACAAAGGGTACAAGCACCATGCCGA
AGATGCTCTGATTATCGCAAATGCCGACTTCATCTTTAAGGAGTGGAAAAAGCTGGACAAAGCCAAGA
AAGTGATGGAGAACCAGATGTTCGAAGAGAAGCAGGCCGAATCTATGCCCGAAATCGAGACAGAACAG
GAGTACAAGGAGATTTTCATCACTCCTCACCAGATCAAGCATATCAAGGATTTCAAGGACTACAAGTA
CTCTCACCGGGTGGATAAAAAGCCCAACAGAGAGCTGATCAATGACACCCTGTATAGTACAAGAAAAG
ACGATAAGGGGAATACCCTGATTGTGAACAATCTGAACGGACTGTACGACAAAGATAATGACAAGCTG
AAAAAGCTGATCAACAAAAGTCCCGAGAAGCTGCTGATGTACCACCATGATCCTCAGACATATCAGAA
ACTGAAGCTGATTATGGAGCAGTACGGCGACGAGAAGAACCCACTGTATAAGTACTATGAAGAGACTG
GGAACTACCTGACCAAGTATAGCAAAAAGGATAATGGCCCCGTGATCAAGAAGATCAAGTACTATGGG
AACAAGCTGAATGCCCATCTGGACATCACAGACGATTACCCTAACAGTCGCAACAAGGTGGTCAAGCT
GTCACTGAAGCCATACAGATTCGATGTCTATCTGGACAACGGCGTGTATAAATTTGTGACTGTCAAGA
ATCTGGATGTCATCAAAAAGGAGAACTACTATGAAGTGAATAGCAAGTGCTACGAAGAGGCTAAAAAG
CTGAAAAAGATTAGCAACCAGGCAGAGTTCATCGCCTCCTTTTACAACAACGACCTGATTAAGATCAA
TGGCGAACTGTATAGGGTCATCGGGGTGAACAATGATCTGCTGAACCGCATTGAAGTGAATATGATTG
ACATCACTTACCGAGAGTATCTGGAAAACATGAATGATAAGCGCCCCCCTCGAATTATCAAAACAATT
GCCTCTAAGACTCAGAGTATCAAAAAGTACTCAACCGACATTCTGGGAAACCTGTATGAGGTGAAGAG
CAAAAAGCACCCTCAGATTATCAAAAAGGGCagcggaggcaagcgtcctgctgctactaagaaagctg
gtcaagctaagaaaaagaaaggatcctacccatacgatgttccagattacgcttaagaattcctagag
ctcgctgatcagcctcgactgtgccttctagttgccagccatctgttgtttgcccctcccccgtgcct
tccttgaccctggaaggtgccactcccactgtcctttcctaataaaatgaggaaattgcatcgcattg
gagggttaattgcgcgcttggcgtaatcatggtcatagctgtttcctgtgtgaaattgttatccgctc
acaattccacacaacatacgagccggaagcataaagtgtaaagcctggggtgcctaatgagtgagcta
actcacattaattgcgttgcgctcactgcccgctttccagtcgggaaacctgtcgtgccagctgcatt
gactcgctgcgctcggtcgttcggctgcggcgagcggtatcagctcactcaaaggcggtaatacggtt
atccacagaatcaggggataacgcaggaaagaacatgtgagcaaaaggccagcaaaaggccaggaacc
gtaaaaaggccgcgttgctggcgtttttccataggctccgcccccctgacgagcatcacaaaaatcga
cgctcaagtcagaggtggcgaaacccgacaggactataaagataccaggcgtttccccctggaagctc
cctcgtgcgctctcctgttccgaccctgccgcttaccggatacctgtccgcctttctcccttcgggaa
gcgtggcgctttctcatagctcacgctgtaggtatctcagttcggtgtaggtcgttcgctccaagctg
ggctgtgtgcacgaaccccccgttcagcccgaccgctgcgccttatccggtaactatcgtcttgagtc
caacccggtaagacacgacttatcgccactggcagcagccactggtaacaggattagcagagcgaggt
atgtaggcggtgctacagagttcttgaagtggtggcctaactacggctacactagaaggacagtattt
ggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctc
ttggtcatgagattatcaaaaaggatcttcacctagatccttttaaattaaaaatgaagttttaaatc
cagcgatctgtctatttcgttcatccatagttgcctgactccccgtcgtgtagataactacgatacgg
atcagcaataaaccagccagccggaagggccgagcgcagaagtggtcctgcaactttatccgcctcca
tccagtctattaattgttgccgggaagctagagtaagtagttcgccagttaatagtttgcgcaacgtt
gttgccattgctacaggcatcgtggtgtcacgctcgtcgtttggtatggcttcattcagctccggttc
ccaacgatcaaggcgagttacatgatcccccatgttgtgcaaaaaagcggttagctccttcggtcctc
cgatcgttgtcagaagtaagttggccgcagtgttatcactcatggttatggcagcactgcataattct
cttactgtcatgccatccgtaagatgcttttctgtgactggtgagtactcaaccaagtcattctgaga
atagtgtatgcggcgaccgagttgctcttgcccggcgtcaatacgggataataccgcgccacatagca
gaactttaaaagtgctcatcattggaaaacgttcttcggggcgaaaactctcaaggatcttaccgctg
ttgagatccagttcgatgtaacccactcgtgcacccaactgatcttcagcatcttttactttcaccag
cgtttctgggtgagcaaaaacaggaaggcaaaatgccgcaaaaaagggaataagggcgacacggaaat
gttgaatactcatactcttcctttttcaatattattgaagcatttatcagggttattgtctcatgagc
ggatacatatttgaatgtatttagaaaaataaacaaataggggttccgcgcacatttccccgaaaagt
gccac
SEQ ID NO: 33
Human p300 (with L553M mutation) protein
MAENVVEPGPPSAKRPKLSSPALSASASDGTDFGSLEDLEHDLPDELINSTELGLINGGDIN
QLQTSLGMVQDAASKHKQLSELLRSGSSPNLNMGVGGPGQVMASQAQQSSPGLGLINSMVKS
PMTQAGLTSPNMGMGTSGPNQGPTQSTGMMNSPVNQPAMGMNTGMNAGMNPGMLAAGNGQGI
MPNQVMNGSIGAGRGRONMQYPNPGMGSAGNLLTEPLQQGSPQMGGQTGLRGPQPLKMGMMN
NPNPYGSPYTQNPGQQIGASGLGLQIQTKTVLSNNLSPFAMDKKAVPGGGMPNMGQQPAPQV
QQPGLVTPVAQGMGSGAHTADPEKRKLIQQQLVLLLHAHKCQRREQANGEVRQCNLPHCRTM
KNVLNHMTHCQSGKSCQVAHCASSEQIISHWKNCTRHDCPVCLPLKNAGDKRNQQPILTGAP
VGLGNPSSLGVGQQSAPNLSTVSQIDPSSIERAYAALGLPYQVNQMPTQPQVQAKNQQNQQP
GQSPQGMRPMSNMSASPMGVNGGVGVQTPSLLSDSMLHSAINSQNPMMSENASVPSMGPMPT
AAQPSTTGIRKQWHEDITQDLRNHLVHKLVQAIFPTPDPAALKDRRMENLVAYARKVEGDMY
ESANNRAEYYHLLAEKIYKIQKELEEKRETRLQKQNMLPNAAGMVPVSMNPGPNMGQPQPGM
TSNGPLPDPSMIRGSVPNQMMPRITPQSGLNQFGQMSMAQPPIVPRQTPPLQHHGQLAQPGA
LNPPMGYGPRMQQPSNQGQFLPQTQFPSQGMNVTNIPLAPSSGQAPVSQAQMSSSSCPVNSP
IMPPGSQGSHIHCPQLPQPALHQNSPSPVPSRTPTPHHTPPSIGAQQPPATTIPAPVPTPPA
MPPGPQSQALHPPPRQTPTPPTTQLPQQVQPSLPAAPSADQPQQQPRSQQSTAASVPTPTAP
LLPPQPATPLSQPAVSIEGQVSNPPSTSSTEVNSQAIAEKQPSQEVKMEAKMEVDQPEPADT
RQALMPTLEALYRQDPESLPFRQPVDPQLLGIPDYFDIVKSPMDLSTIKRKLDTGQYQEPWQ
YVDDIWLMFNNAWLYNRKTSRVYKYCSKLSEVFEQEIDPVMQSLGYCCGRKLEFSPQTLCCY
GKOLCTIPRDATYYSYQNRYHFCEKCFNEIQGESVSLGDDPSQPQTTINKEQFSKRKNDTLD
PELFVECTECGRKMHQICVLHHEIIWPAGFVCDGCLKKSARTRKENKESAKRLPSTRLGTFL
ENRVNDFLRRONHPESGEVTVRVVHASDKTVEVKPGMKARFVDSGEMAESFPYRTKALFAFE
EIDGVDLCFFGMHVQEYGSDCPPPNQRRVYISYLDSVHFFRPKCLRTAVYHEILIGYLEYVK
KLGYTTGHIWACPPSEGDDYIFHCHPPDQKIPKPKRLQEWYKKMLDKAVSERIVHDYKDIEK
QATEDRLTSAKELPYFEGDEWPNVLEESIKELEQEEEERKREENTSNESTDVTKGDSKNAKK
KNNKKTSKNKSSLSRGNKKKPGMPNVSNDLSQKLYATMEKHKEVFFVIRLIAGPAANSLPPI
VDPDPLIPCDLMDGRDAFLTLARDKHLEFSSLRRAQWSTMCMLVELHTQSQDRFVYTCNECK
HHVETRWHCTVCEDYDLCITCYNTKNHDHKMEKLGLGLDDESNNQQAAATQSPGDSRRLSIQ
RCIQSLVHACQCRNANCSLPSCQKMKRVVQHTKGCKRKINGGCPICKQLIALCCYHAKHCQE
NKCPVPFCLNIKQKLRQQQLQHRLQQAQMLRRRMASMQRTGVVGQQQGLPSPTPATPTTPTG
QQPTTPQTPQPTSQPQPTPPNSMPPYLPRTQAAGPVSQGKAAGQVTPPTPPQTAQPPLPGPP
PAAVEMAMQIQRAAETQROMAHVQIFORPIQHQMPPMTPMAPMGMNPPPMTRGPSGHLEPGM
GPTGMQQQPPWSQGGLPQPQQLQSGMPRPAMMSVAQHGQPLNMAPQPGLGQVGISPLKPGTV
SQQALQNLLRTLRSPSSPLQQQQVLSILHANPQLLAAFIKQRAAKYANSNPQPIPGQPGMPQ
GQPGLQPPTMPGQQGVHSNPAMQNMNPMQAGVQRAGLPQQQPQQQLQPPMGGMSPQAQQMNM
NHNTMPSQFRDILRRQQMMQQQQQQGAGPGIGPGMANHNQFQQPQGVGYPPQQQQRMQHHMQ
QMQQGNMGQIGQLPQALGAEAGASLQAYQQRLLQQQMGSPVQPNPMSPQQHMLPNQAQSPHL
QGQQIPNSLSNQVRSPQPVPSPRPQSQPPHSSPSPRMQPQPSPHHVSPQTSSPHPGLVAAQA
NPMEQGHFASPDQNSMLSQLASNPGMANLHGASATDLGLSTDNSDLNSNLSQSTLDIH
SEQ ID NO: 34
Human p300 Core Effector protein (aa 1048-1664 of SEQ ID NO: 33)
IFKPEELROALMPTLEALYRQDPESLPFRQPVDPQLLGIPDYFDIVKSPMDLSTIKRKLDTG
QYQEPWQYVDDIWLMFNNAWLYNRKTSRVYKYCSKLSEVFEQEIDPVMQSLGYCCGRKLEFS
PQTLCCYGKQLCTIPRDATYYSYQNRYHFCEKCFNEIQGESVSLGDDPSQPQTTINKEQFSK
RKNDTLDPELFVECTECGRKMHQICVLHHEIIWPAGEVCDGCLKKSARTRKENKFSAKRLPS
TRLGTFLENRVNDFLRRONHPESGEVTVRVVHASDKTVEVKPGMKAREVDSGEMAESFPYRT
KALFAFEEIDGVDLCFFGMHVQEYGSDCPPPNQRRVYISYLDSVHFFRPKCLRTAVYHEILI
GYLEYVKKLGYTTGHIWACPPSEGDDYIFHCHPPDQKIPKPKRLQEWYKKMLDKAVSERIVH
DYKDIFKOATEDRLTSAKELPYFEGDFWPNVLEESIKELEQEEEERKREENTSNESTDVTKG
DSKNAKKKNNKKTSKNKSSLSRGNKKKPGMPNVSNDLSQKLYATMEKHKEVFFVIRLIAGPA
ANSLPPIVDPDPLIPCDLMDGRDAFLTLARDKHLEFSSLRRAQWSTMCMLVELHTQSQD
SEQ ID NO: 35
VP64-dCas9-VP64 protein
RADALDDFDLDMLGSDALDDFDLDMLGSDALDDFDLDMLGSDALDDFDLDMVNPKKKRKVGR
GMDKKYSIGLAIGTNSVGWAVITDEYKVPSKKFKVLGNTDRHSIKKNLIGALLEDSGETAEA
TRLKRTARRRYTRRKNRICYLQEIFSNEMAKVDDSFFHRLEESFLVEEDKKHERHPIFGNIV
DEVAYHEKYPTIYHLRKKLVDSTDKADLRLIYLALAHMIKFRGHFLIEGDLNPDNSDVDKLF
IQLVQTYNQLFEENPINASGVDAKAILSARLSKSRRLENLIAQLPGEKKNGLFGNLIALSLG
LTPNFKSNFDLAEDAKLQLSKDTYDDDLDNILAQIGDQYADLFLAAKNLSDAILLSDILRVN
TEITKAPLSASMIKRYDEHHQDLTLLKALVRQQLPEKYKEIFFDQSKNGYAGYIDGGASQEE
FYKFIKPILEKMDGTEELLVKLNREDLLRKQRTFDNGSIPHQIHLGELHAILERQEDFYPFL
KDNREKIEKILTERIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLEKTNR
KVTVKQLKEDYFKKIECFDSVEISGVEDRFNASLGTYHDLLKIIKDKDFLDNEENEDILEDI
VLTLTLFEDREMIEERLKTYAHLFDDKVMKQLKERRYTGWGRLSRKLINGIRDKQSGKTILD
FLKSDGFANRNEMQLIHDDSLTFKEDIQKAQVSGQGDSLHEHIANLAGSPAIKKGILQTVKV
VDELVKVMGRHKPENIVIEMARENQTTQKGQKNSRERMKRIEEGIKELGSQILKEHPVENTQ
LQNEKLYLYYLONGRDMYVDQELDINRLSDYDVDAIVPQSFLKDDSIDNKVLTRSDKNRGKS
DNVPSEEVVKKMKNYWRQLLNAKLITQRKFDNLTKAERGGLSELDKAGFIKROLVETRQITK
HVAQILDSRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESILPKRN
SDKLIARKKDWDPKKYGGFDSPTVAYSVLVVAKVEKGKSKKLKSVKELLGITIMERSSFEKN
PIDFLEAKGYKEVKKDLIIKLPKYSLFELENGRKRMLASAGELQKGNELALPSKYVNFLYLA
SHYEKLKGSPEDNEQKQLFVEQHKHYLDEIIEQISEFSKRVILADANLDKVLSAYNKHRDKP
IREQAENIIHLETLTNLGAPAAFKYFDTTIDRKRYTSTKEVLDATLIHQSITGLYETRIDLS
QLGGDSRADPKKKRKVASRADALDDFDLDMLGSDALDDFDLDMLGSDALDDFDLDMLGSDAL
DDFDLDMLI
SEQ ID NO: 36
VP64-dCas9-VP64 DNA
cgggctgacgcattggacgattttgatctggatatgctgggaagtgacgccctcgatgattt
tgaccttgacatgcttggttcggatgcccttgatgactttgacctcgacatgctcggcagtg
acgcccttgatgatttcgacctggacatggttaaccccaagaagaagaggaaggtgggccgc
ggaatggacaagaagtactccattgggctcgccatcggcacaaacagcgtcggctgggccgt
cattacggacgagtacaaggtgccgagcaaaaaattcaaagttctgggcaataccgatcgcc
acagcataaagaagaacctcattggcgccctcctgttcgactccggggaaaccgccgaagcc
acgcggctcaaaagaacagcacggcgcagatatacccgcagaaagaatcggatctgctacct
gcaggagatctttagtaatgagatggctaaggtggatgactctttcttccataggctggagg
agtcctttttggtggaggaggataaaaagcacgagcgccacccaatctttggcaatatcgtg
gacgaggtggcgtaccatgaaaagtacccaaccatatatcatctgaggaagaagcttgtaga
cagtactgataaggctgacttgcggttgatctatctcgcgctggcgcatatgatcaaatttc
ggggacacttcctcatcgagggggacctgaacccagacaacagcgatgtcgacaaactcttt
atccaactggttcagacttacaatcagcttttcgaagagaacccgatcaacgcatccggagt
tgacgccaaagcaatcctgagcgctaggctgtccaaatcccggcggctcgaaaacctcatcg
cacagctccctggggagaagaagaacggcctgtttggtaatcttatcgccctgtcactcggg
ctgacccccaactttaaatctaacttcgacctggccgaagatgccaagcttcaactgagcaa
agacacctacgatgatgatctcgacaatctgctggcccagatcggcgaccagtacgcagacc
acggagatcaccaaagctccgctgagcgctagtatgatcaagcgctatgatgagcaccacca
agacttgactttgctgaaggcccttgtcagacagcaactgcctgagaagtacaaggaaattt
tcttcgatcagtctaaaaatggctacgccggatacattgacggcggagcaagccaggaggaa
ttttacaaatttattaagcccatcttggaaaaaatggacggcaccgaggagctgctggtaaa
gcttaacagagaagatctgttgcgcaaacagcgcactttcgacaatggaagcatcccccacc
agattcacctgggcgaactgcacgctatcctcaggcggcaagaggatttctacccctttttg
aaagataacagggaaaagattgagaaaatcctcacatttcggataccctactatgtaggccc
cctcgcccggggaaattccagattcgcgtggatgactcgcaaatcagaagagaccatcactc
cctggaacttcgaggaagtcgtggataagggggcctctgcccagtccttcatcgaaaggatg
actaactttgataaaaatctgcctaacgaaaaggtgcttcctaaacactctctgctgtacga
gtacttcacagtttataacgagctcaccaaggtcaaatacgtcacagaagggatgagaaagc
cagcattcctgtctggagagcagaagaaagctatcgtggacctcctcttcaagacgaaccgg
aaagttaccgtgaaacagctcaaagaagactatttcaaaaagattgaatgtttcgactctgt
tgaaatcagcggagtggaggatcgcttcaacgcatccctgggaacgtatcacgatctcctga
aaatcattaaagacaaggacttcctggacaatgaggagaacgaggacattcttgaggacatt
gtcctcacccttacgttgtttgaagatagggagatgattgaagaacgcttgaaaacttacgc
tcatctcttcgacgacaaagtcatgaaacagctcaagaggcgccgatatacaggatgggggc
ggctgtcaagaaaactgatcaatgggatccgagacaagcagagtggaaagacaatcctggat
tttcttaagtccgatggatttgccaaccggaacttcatgcagttgatccatgatgactctct
cacctttaaggaggacatccagaaagcacaagtttctggccagggggacagtcttcacgagc
acatcgctaatcttgcaggtagcccagctatcaaaaagggaatactgcagaccgttaaggtc
gtggatgaactcgtcaaagtaatgggaaggcataagcccgagaatatcgttatcgagatggc
ccgagagaaccaaactacccagaagggacagaagaacagtagggaaaggatgaagaggattg
cttcagaatgagaagctctacctgtactacctgcagaacggcagggacatgtacgtggatca
ggaactggacatcaatcggctctccgactacgacgtggatgccatcgtgccccagtcttttc
tcaaagatgattctattgataataaagtgttgacaagatccgataaaaatagagggaagagt
gataacgtcccctcagaagaagttgtcaagaaaatgaaaaattattggcggcagctgctgaa
cgccaaactgatcacacaacggaagttcgataatctgactaaggctgaacgaggtggcctgt
ctgagttggataaagccggcttcatcaaaaggcagcttgttgagacacgccagatcaccaag
cacgtggcccaaattctcgattcacgcatgaacaccaagtacgatgaaaatgacaaactgat
tcgagaggtgaaagttattactctgaagtctaagctggtctcagatttcagaaaggactttc
agttttataaggtgagagagatcaacaattaccaccatgcgcatgatgcctacctgaatgca
gtggtaggcactgcacttatcaaaaaatatcccaagcttgaatctgaatttgtttacggaga
ctataaagtgtacgatgttaggaaaatgatcgcaaagtctgagcaggaaataggcaaggcca
ccgctaagtacttcttttacagcaatattatgaattttttcaagaccgagattacactggcc
aatggagagattcggaagcgaccacttatcgaaacaaacggagaaacaggagaaatcgtgtg
ggacaagggtagggatttcgcgacagtccggaaggtcctgtccatgccgcaggtgaacatcg
ttaaaaagaccgaagtacagaccggaggcttctccaaggaaagtatcctcccgaaaaggaac
agcgacaagctgatcgcacgcaaaaaagattgggaccccaagaaatacggcggattcgattc
tcctacagtcgcttacagtgtactggttgtggccaaagtggagaaagggaagtctaaaaaac
tcaaaagcgtcaaggaactgctgggcatcacaatcatggagcgatcaagcttcgaaaaaaac
cccatcgactttctcgaggcgaaaggatataaagaggtcaaaaaagacctcatcattaagct
tcccaagtactctctctttgagcttgaaaacggccggaaacgaatgctcgctagtgcgggcg
agctgcagaaaggtaacgagctggcactgccctctaaatacgttaatttcttgtatctggcc
agccactatgaaaagctcaaagggtctcccgaagataatgagcagaagcagctgttcgtgga
acaacacaaacactaccttgatgagatcatcgagcaaataagcgaattctccaaaagagtga
tcctcgccgacgctaacctcgataaggtgctttctgcttacaataagcacagggataagccc
atcagggagcaggcagaaaacattatccacttgtttactctgaccaacttgggcgcgcctgc
agccttcaagtacttcgacaccaccatagacagaaagcggtacacctctacaaaggaggtcc
tggacgccacactgattcatcagtcaattacggggctctatgaaacaagaatcgacctctct
cagctcggtggagacagcagggctgaccccaagaagaagaggaaggtggctagccgcgccga
cgcgctggacgatttcgatctcgacatgctgggttctgatgccctcgatgactttgacctgg
atatgttgggaagcgacgcattggatgactttgatctggacatgctcggctccgatgctctg
gacgatttcgatctcgatatgttaatc
SEQ ID NO: 37
Streptococcus pyogenes dCas9-KRAB protein
MDYKDHDGDYKDHDIDYKDDDDKMAPKKKRKVGRGMDKKYSIGLAIGTNSVGWAVITDEYKV
PSKKEKVLGNTDRHSIKKNLIGALLFDSGETAEATRLKRTARRRYTREKNRICYLQEIFSNE
MAKVDDSFFHRLEESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPINASGVDAKAILS
ARLSKSRRLENDIAQLPGEKKNGLFGNLIALSLGLTPNFKSNFDLAEDAKLQLSKDTYDDDL
DNILAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLL
RKQRTFDNGSIPHQIHLGELHAILRRQEDFYPFLKDNREKIEKILTFRIPYYVGPLARGNSR
FAWMTRKSEETITPWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKIECFDSVEISGVED
RENASLGTYHDLLKIIKDKDFLDNEENEDILEDIVLTLTLFEDREMIEERLKTYAHLFDDKV
MKQLKRRRYTGWGRLSRKLINGIRDKQSGKTILDFLKSDGFANRNEMQLIHDDSLTFKEDIQ
KAQVSGQGDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLONGRDMYVDQELDINRL
SDYDVDAIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVIT
LKSKLVSDERKDFQFYKVREINNYHHAHDAYLNAVVGTALIKKYPKLESEFVYGDYKVYDVR
KMIAKSEQEIGKATAKYFFYSNIMNFFKTEITLANGEIRKRPLIETNGETGEIVWDKGRDFA
TVRKVLSMPQVNIVKKTEVQTGGFSKESILPKRNSDKLIARKKDWDPKKYGGFDSPTVAYSV
LVVAKVEKGKSKKLKSVKELLGITIMERSSFEKNPIDFLEAKGYKEVKKDLIIKLPKYSLFE
EITEQISEFSKRVILADANLDKVLSAYNKHRDKPIREQAENIIHLETLTNLGAPAAFKYFDT
TIDRKRYTSTKEVLDATLIHQSITGLYETRIDLSQLGGDSRADPKKKRKVASDAKSLTAWSR
TLVTFKDVFVDFTREEWKLLDTAQQILYRNVMLENYKNLVSLGYQLTKPDVILRLEKGEEPW
LVEREIHQETHPDSETAFEIKSSVPKKKRKV
SEQ ID NO: 38
Polynucleotide encoding Streptococcus pyogenes dCas9-KRAB
atggactacaaagaccatgacggtgattataaagatcatgacatcgattacaaggatgacga
tgacaagatggcccccaagaagaagaggaaggtgggccgcggaatggacaagaagtactcca
ttgggctcgccatcggcacaaacagcgtcggctgggccgtcattacggacgagtacaaggtg
ccgagcaaaaaattcaaagttctgggcaataccgatcgccacagcataaagaagaacctcat
tggcgccctcctgttcgactccggggaaaccgccgaagccacgcggctcaaaagaacagcac
ggcgcagatatacccgcagaaagaatcggatctgctacctgcaggagatctttagtaatgag
atggctaaggtggatgactctttcttccataggctggaggagtcctttttggtggaggagga
taaaaagcacgagcgccacccaatctttggcaatatcgtggacgaggtggcgtaccatgaaa
agtacccaaccatatatcatctgaggaagaagcttgtagacagtactgataaggctgacttg
cggttgatctatctcgcgctggcgcatatgatcaaatttcggggacacttcctcatcgaggg
ggacctgaacccagacaacagcgatgtcgacaaactctttatccaactggttcagacttaca
atcagcttttcgaagagaacccgatcaacgcatccggagttgacgccaaagcaatcctgagc
gctaggctgtccaaatcccggcggctcgaaaacctcatcgcacagctccctggggagaagaa
gaacggcctgtttggtaatcttatcgccctgtcactcgggctgacccccaactttaaatcta
acttcgacctggccgaagatgccaagcttcaactgagcaaagacacctacgatgatgatctc
gacaatctgctggcccagatcggcgaccagtacgcagacctttttttggcggcaaagaacct
gtcagacgccattctgctgagtgatattctgcgagtgaacacggagatcaccaaagctccgc
tgagcgctagtatgatcaagcgctatgatgagcaccaccaagacttgactttgctgaaggcc
cttgtcagacagcaactgcctgagaagtacaaggaaattttcttcgatcagtctaaaaatgg
ctacgccggatacattgacggcggagcaagccaggaggaattttacaaatttattaagccca
tcttggaaaaaatggacggcaccgaggagctgctggtaaagcttaacagagaagatctgttg
cgcaaacagcgcactttcgacaatggaagcatcccccaccagattcacctgggcgaactgca
cgctatcctcaggcggcaagaggatttctacccctttttgaaagataacagggaaaagattg
agaaaatcctcacatttcggataccctactatgtaggccccctcgcccggggaaattccaga
ttcgcgtggatgactcgcaaatcagaagagaccatcactccctggaacttcgaggaagtcgt
ggataagggggcctctgcccagtccttcatcgaaaggatgactaactttgataaaaatctgc
ctaacgaaaaggtgcttcctaaacactctctgctgtacgagtacttcacagtttataacgag
ctcaccaaggtcaaatacgtcacagaagggatgagaaagccagcattcctgtctggagagca
gaagaaagctatcgtggacctcctcttcaagacgaaccggaaagttaccgtgaaacagctca
aagaagactatttcaaaaagattgaatgtttcgactctgttgaaatcagcggagtggaggat
cgcttcaacgcatccctgggaacgtatcacgatctcctgaaaatcattaaagacaaggactt
cctggacaatgaggagaacgaggacattcttgaggacattgtcctcacccttacgttgtttg
aagatagggagatgattgaagaacgcttgaaaacttacgctcatctcttcgacgacaaagtc
atgaaacagctcaagaggcgccgatatacaggatgggggcggctgtcaagaaaactgatcaa
tgggatccgagacaagcagagtggaaagacaatcctggattttcttaagtccgatggatttg
ccaaccggaacttcatgcagttgatccatgatgactctctcacctttaaggaggacatccag
aaagcacaagtttctggccagggggacagtcttcacgagcacatcgctaatcttgcaggtag
cccagctatcaaaaagggaatactgcagaccgttaaggtcgtggatgaactcgtcaaagtaa
tgggaaggcataagcccgagaatatcgttatcgagatggcccgagagaaccaaactacccag
aagggacagaagaacagtagggaaaggatgaagaggattgaagagggtataaaagaactggg
gtcccaaatccttaaggaacacccagttgaaaacacccagcttcagaatgagaagctctacc
tctactacctgcagaacggcagggacatgtacgtggatcaggaactggacatcaatcggctc
tccgactacgacgtggatgccatcgtgccccagtcttttctcaaagatgattctattgataa
taaagtgttgacaagatccgataaaaatagagggaagagtgataacgtcccctcagaagaag
ttgtcaagaaaatgaaaaattattggcggcagctgctgaacgccaaactgatcacacaacgg
aagttcgataatctgactaaggctgaacgaggtggcctgtctgagttggataaagccggctt
catcaaaaggcagcttgttgagacacgccagatcaccaagcacgtggcccaaattctcgatt
cacgcatgaacaccaagtacgatgaaaatgacaaactgattcgagaggtgaaagttattact
ctgaagtctaagctggtctcagatttcagaaaggactttcagttttataaggtgagagagat
caacaattaccaccatgcgcatgatgcctacctgaatgcagtggtaggcactgcacttatca
aaaaatatcccaagcttgaatctgaatttgtttacggagactataaagtgtacgatgttagg
aaaatgatcgcaaagtctgagcaggaaataggcaaggccaccgctaagtacttcttttacag
caatattatgaattttttcaagaccgagattacactggccaatggagagattcggaagcgac
cacttatcgaaacaaacggagaaacaggagaaatcgtgtgggacaagggtagggatttcgcg
cggaggcttctccaaggaaagtatcctcccgaaaaggaacagcgacaagctgatcgcacgca
aaaaagattgggaccccaagaaatacggcggattcgattctcctacagtcgcttacagtgta
ctggttgtggccaaagtggagaaagggaagtctaaaaaactcaaaagcgtcaaggaactgct
gggcatcacaatcatggagcgatcaagcttcgaaaaaaaccccatcgactttctcgaggcga
aaggatataaagaggtcaaaaaagacctcatcattaagcttcccaagtactctctctttgag
cttgaaaacggccggaaacgaatgctcgctagtgcgggcgagctgcagaaaggtaacgagct
ggcactgccctctaaatacgttaatttcttgtatctggccagccactatgaaaagctcaaag
ggtctcccgaagataatgagcagaagcagctgttcgtggaacaacacaaacactaccttgat
gagatcatcgagcaaataagcgaattctccaaaagagtgatcctcgccgacgctaacctcga
taaggtgctttctgcttacaataagcacagggataagcccatcagggagcaggcagaaaaca
ttatccacttgtttactctgaccaacttgggcgcgcctgcagccttcaagtacttcgacacc
accatagacagaaagcggtacacctctacaaaggaggtcctggacgccacactgattcatca
gtcaattacggggctctatgaaacaagaatcgacctctctcagctcggtggagacagcaggg
ctgaccccaagaagaagaggaaggtggctagcgatgctaagtcactgactgcctggtcccgg
acactggtgaccttcaaggatgtgtttgtggacttcaccagggaggagtggaagctgctgga
cactgctcagcagatcctgtacagaaatgtgatgctggagaactataagaacctggtttcct
tgggttatcagcttactaagccagatgtgatcctccggttggagaagggagaagagccctgg
ctggtggagagagaaattcaccaagagacccatcctgattcagagactgcatttgaaatcaa
atcatcagttccgaaaaagaaacgcaaagtttga
SEQ ID NO: 39
DNA sequence of the gRNA constant region
gtttaagagctatgotggaaacagcatagcaagtttaaataaggctagtccgttatcaactt
gaaaaagtggcaccgagtcggtgc
SEQ ID NO: 40
RNA sequence of the gRNA constant region
guuuaagagcuaugcuggaaacagcauagcaaguuuaaauaaggcuaguccquuaucaacuu
gaaaaaguggcaccgagucggugc

| Gene | | | |
|---|---|---|---|
| Targeted | gRNA ID | gRNA targeted sequence | gRNA sequence |
| OGDH | OGDH.1 | gactagttcgaactatgtgg | gacuaguucgaacuaugugg |
| | | (SEQ ID NO: 41) | (SEQ ID NO: 57) |
| OGDH | OGDH.2 | gatcatgcagttcacaaatg | gaucaugcaguucacaaaug |
| | | (SEQ ID NO: 42) | (SEQ ID NO: 58) |
| OGDH | OGDH.3 | gttggccactcatagatacg | guuggccacucauagauacg |
| | | (SEQ ID NO: 43) | (SEQ ID NO: 59) |
| OGDH | OGDH.4 | ttcctgtcccccgatgaaag | uuccugucccccgaugaaag |
| | | (SEQ ID NO: 44) | (SEQ ID NO: 60) |
| LIPT1 | LIPT1.1 | agtacttcacaggtcagagt | aquacuucacaggucagagu |
| | | (SEQ ID NO: 45) | (SEQ ID NO: 61) |
| LIPT1 | LIPT1.2 | atgcctaccaattacaacag | augccuaccaauuacaacag |
| | | (SEQ ID NO: 46) | (SEQ ID NO: 62) |
| LIPT1 | LIPT1.3 | gaacagcttctaagatcggc | gaacagcuucuaagaucggc |
| | | (SEQ ID NO: 47) | (SEQ ID NO: 63) |
| LIPT1 | LIPT1.4 | ggaacagtctaccatgatat | ggaacagucuaccaugauau |
| | | (SEQ ID NO: 48) | (SEQ ID NO: 64) |
| SDHC | SDHC.1 | aagcaataccagtgccacgg | aagcaauaccagugccacgg |
| | | (SEQ ID NO: 49) | (SEQ ID NO: 65) |
| SDHC | SDHC.2 | cggccaaagaagagatggag | cggccaaagaagagauggag |
| | | (SEQ ID NO: 50) | (SEQ ID NO: 66) |
| SDHC | SDHC.3 | gccaaaaagagagacccctg | gccaaaaagagagaccccug |
| | | (SEQ ID NO: 51) | (SEQ ID NO: 67) |
| SDHC | SDHC.4 | tacctgtagatagtaatgtg | uaccuguagauaguaaugug |
| | | (SEQ ID NO: 52) | (SEQ ID NO: 68) |
| DHRS7B | DHRS7B.1 | aatgctgggatcagctaccg | aaugcugggaucagcuaccg |
| | | (SEQ ID NO: 53) | (SEQ ID NO: 69) |
| DHRS7B | DHRS7B.2 | cccagagtetgtgaggtcga | cccagagucugugaggucga |
| | | (SEQ ID NO: 54) | (SEQ ID NO: 70) |
| DHRS7B | DHRS7B.3 | ggtgctaaactggtgctctg | ggugcuaaacuggugcucug |
| | | (SEQ ID NO: 55) | (SEQ ID NO: 71) |
| DHRS7B | DHRS7B.4 | tgetgctgatggcgacaatg | ugcugcugauggegacaaug |
| | | (SEQ ID NO: 56) | (SEQ ID NO: 72) |

1. A composition for treating a subject having Prader Willi Syndrome (PWS) or a PWS-like disorder, the composition comprising an inhibitor of a gene selected from OGDH, LIPT1, SDHC, and DHRS7B.

2. A composition for activating SNRPN, SPA1, SPA2, or SNORD116, or a combination thereof, the composition comprising an inhibitor of a gene selected from OGDH, LIPT1, SDHC, and DHRS7B.

3. The composition of claim 1 or 2, wherein the composition reduces expression of the gene selected from OGDH, LIPT1, SDHC, and DHRS7B, or wherein the composition reduces an activity of a protein encoded by the gene selected from OGDH, LIPT1, SDHC, and DHRS7B.

4. The composition of one of claims 1-3, wherein the inhibitor comprises a small molecule, a polynucleotide, a polypeptide, or a combination thereof.

5. The composition of any one of claims 1-4, wherein the polynucleotide comprises an inhibitory nucleic acid selected from an antisense oligonucleotide, siRNA, RNAi, shRNA, LNA, and PNA.

6. The composition of claim 5, wherein the inhibitory nucleic acid comprises one or more of a modified internucleoside linkage, a modified sugar moiety, and/or a modified nucleobase.

7. The composition of any one of claims 1-4, wherein the inhibitor comprises an antibody.

8. The composition of any one of claims 1-4, wherein the inhibitor comprises a DNA Targeting System.

9. The composition of claim 8, wherein the DNA Targeting System comprises:
(a) a zinc finger protein or TALE or DNA binding fusion protein that targets the gene selected from OGDH, LIPT1, SDHC, and DHRS7B; or
(b) a CRISPR/Cas9 system that targets the gene selected from OGDH, LIPT1, SDHC, and DHRS7B.

10. The composition of claim 9, wherein the DNA binding fusion protein comprises a zinc finger protein or TALE, and a polypeptide domain having transcription repression activity and/or nuclease activity.

11. The composition of claim 10, wherein the polypeptide domain having transcription repression activity comprises KRAB, MECP2, Mad mSIN3 interaction domain (SID), ERF repressor domain (ERD), SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, and/or CTCF.

12. The composition of claim 10, wherein the polypeptide domain having nuclease activity comprises FokI.

13. The composition of claim 9, wherein the CRISPR/Cas9 system comprises:
(a) a Cas9 protein or a fusion protein comprising the Cas9 protein; and
(b) a gRNA targeting the gene selected from OGDH, LIPT1, SDHC, and DHRS7B, or a portion thereof.

14. The composition of claim 13, wherein the Cas9 protein is a Streptococcus pyogenes Cas9 protein or a Staphylococcus aureus Cas9 protein.

15. The composition of claim 14, wherein the Streptococcus pyogenes Cas9 protein comprises the polypeptide sequence of SEQ ID NO: 18, and wherein the Staphylococcus aureus Cas9 protein comprises the polypeptide sequence of SEQ ID NO: 19.

16. The composition of claim 13, wherein the Cas9 protein is nuclease-deficient dCas9 and comprises the polypeptide sequence of SEQ ID NO: 20 or 21 or is encoded by a polynucleotide sequence comprising SEQ ID NO: 22 or 23.

17. The composition of any one of claims 13-16, wherein the fusion protein comprises two heterologous polypeptide domains, wherein the first polypeptide domain comprises the Cas9 protein, and wherein the second polypeptide domain has an activity selected from transcription activation activity, transcription repression activity, transcription release factor activity, histone modification activity, nuclease activity, nucleic acid association activity, methylase activity, and demethylase activity.

18. The composition of claim 17, wherein the second polypeptide domain has transcription repression activity and/or nuclease activity.

19. The composition of claim 18, wherein the second polypeptide domain comprises KRAB, MECP2, Mad mSIN3 interaction domain (SID), ERF repressor domain (ERD), SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, and/or FokI.

20. The composition of claim 19, wherein the fusion protein comprises dCas9-KRAB.

21. The composition of any one of claims 13-20, wherein the gene is OGDH and the gRNA binds and targets a polynucleotide sequence selected from SEQ ID NOs: 41-44, or
wherein the gene is LIPT1 and the gRNA binds and targets a polynucleotide sequence selected from SEQ ID NOs: 45-48, or
wherein the gene is SDHC and the gRNA binds and targets a polynucleotide sequence selected from SEQ ID NOs: 49-52, or
wherein the gene is DHRS7B and the gRNA binds and targets a polynucleotide sequence selected from SEQ ID NOs: 53-56.

22. A guide RNA (gRNA) that binds and targets a polynucleotide sequence selected from SEQ ID NOs: 41-58, a complement thereof, a variant thereof, or fragment thereof, or that comprises a polynucleotide sequence selected from SEQ ID NOs: 57-72, a complement thereof, a variant thereof, or fragment thereof.

23. A polynucleotide encoding the composition of any one of claims 1-22 or at least one component thereof, or the gRNA of claim 22.

24. A vector comprising the polynucleotide of claim 23.

25. The vector of claim 24, wherein the vector is a viral vector.

26. The vector of claim 24, wherein the vector is a retroviral vector, lentiviral vector, adenoviral vector, adeno-associated virus (AAV) vector, synthetic vector, or vector encapsulated within a lipid nanoparticle.

27. A pharmaceutical composition comprising the composition of any one of claims 1-21 or at least one component thereof, the gRNA of claim 22, the polynucleotide of claim 23, or the vector of any one of claims 24-26, or a combination thereof.

28. A method of treating a subject having PWS or a PWS-like disorder, the method comprising administering to the subject the pharmaceutical composition of claim 27.

29. The method of claim 28, wherein the subject has a PWS Type 1 large deletion, a PWS Type 2 large deletion, a PWS imprinting center mutation, PWS uniparental disomy, a PWS microdeletion encompassing SNORD116 but not MAGEL2, a PWS or PWS-like atypical deletion encompassing MAGEL2 but not SNORD116, heterozygous Schaaf-Yang syndrome, or MAGEL2 disorder.

30. The method of any one of claims 28-29, wherein expression of a gene within the maternal copy of the 15q11-13 locus is increased in the subject.

31. A method of activating a gene selected from SNRPN, SPA1, SPA2, and SNORD116, or a combination thereof, in a subject in need thereof, the method comprising administering to the subject the pharmaceutical composition of claim 27.


SEQUENCE LISTING (72 sequences)

SEQ ID NO: 1 - DNA, 3 bp, Artificial Sequence
  Synthetic
  misc_feature (1)..(1): n is a, c, g, or t
  r (2)..(2): r is a, or g
nrg

SEQ ID NO: 2 - DNA, 3 bp, Artificial Sequence
  Synthetic
  misc_feature (1)..(1): n is a, c, g, or t
ngg

SEQ ID NO: 3 - DNA, 3 bp, Artificial Sequence
  Synthetic
  misc_feature (1)..(1): n is a, c, g, or t
nag

SEQ ID NO: 4 - DNA, 5 bp, Artificial Sequence
  Synthetic
  misc_feature (1)..(1): n is a, c, g, or t
  misc_feature (4)..(4): n is a, c, g, or t
nggng

SEQ ID NO: 5 - DNA, 7 bp, Artificial Sequence
  Synthetic
  misc_feature (1)..(2): n is a, c, g, or t
  w (7)..(7): w is a, or t
nnagaaw

SEQ ID NO: 6 - DNA, 4 bp, Artificial Sequence
  Synthetic
  misc_feature (1)..(1): n is a, c, g, or t
  r (4)..(4): r is a, or g
naar

SEQ ID NO: 7 - DNA, 5 bp, Artificial Sequence
  Synthetic
  misc_feature (1)..(2): n is a, c, g, or t
  r (4)..(5): r is a, or g
nngrr

SEQ ID NO: 8 - DNA, 6 bp, Artificial Sequence
  Synthetic
  misc_feature (1)..(2): n is a, c, g, or t
  r (4)..(5): r is a, or g
  misc_feature (6)..(6): n is a, c, g, or t
nngrrn

SEQ ID NO: 9 - DNA, 6 bp, Artificial Sequence
  Synthetic
  misc_feature (1)..(2): n is a, c, g, or t
  r (4)..(5): r is a, or g
nngrrt

SEQ ID NO: 10 - DNA, 6 bp, Artificial Sequence
  Synthetic
  misc_feature (1)..(2): n is a, c, g, or t
  r (4)..(5): r is a, or g
  v (6)..(6): v is a, or c, or g
nngrrv

SEQ ID NO: 11 - DNA, 8 bp, Artificial Sequence
  Synthetic
  misc_feature (1)..(4): n is a, c, g, or t
nnnngatt

SEQ ID NO: 12 - DNA, 8 bp, Artificial Sequence
  Synthetic
  misc_feature (1)..(4): n is a, c, g, or t
  misc_feature (6)..(8): n is a, c, g, or t
nnnngnnn

SEQ ID NO: 13 - DNA, 3 bp, Artificial Sequence
  Synthetic
  misc_feature (1)..(1): n is a, c, g, or t
nga

SEQ ID NO: 14 - DNA, 6 bp, Artificial Sequence
  Synthetic
  misc_feature (1)..(3): n is a, c, g, or t
  r (4)..(5): r is a, or g
nnnrrt

SEQ ID NO: 15 - DNA, 6 bp, Artificial Sequence
  Synthetic
attcct

SEQ ID NO: 16 - DNA, 4 bp, Artificial Sequence
  Synthetic
  misc_feature (1)..(1): n is a, c, g, or t
  misc_feature (4)..(4): n is a, c, g, or t
ngan

SEQ ID NO: 17 - DNA, 4 bp, Artificial Sequence
  Synthetic
  misc_feature (1)..(1): n is a, c, g, or t
  misc_feature (3)..(3): n is a, c, g, or t
ngng

SEQ ID NO: 18 - protein, 1368 aa, Streptococcus pyogenes [sequence omitted]

SEQ ID NO: 19 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 20 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 21 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 22 - DNA, 3159 bp, Artificial Sequence
  Synthetic
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 23 - DNA, 3159 bp, Artificial Sequence
  Synthetic
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 24 - DNA, 4107 bp, Artificial Sequence
  Synthetic
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 25 - DNA, 3158 bp, Artificial Sequence
  Synthetic
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggcctccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 26 - DNA, 3159 bp, Artificial Sequence
  Synthetic
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 27 - DNA, 3159 bp, Artificial Sequence
  Synthetic
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 28 - DNA, 3255 bp, Artificial Sequence
  Synthetic
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccagagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaggcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 29 - DNA, 3239 bp, Artificial Sequence
  Synthetic
accggtgccaccatgtacccatacgatgttccagattacgcttcgccgaagaaaaagcgcaaggtcgaagcgtccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatcttacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggctaagaa

SEQ ID NO: 30 - DNA, 3255 bp, Artificial Sequence
  Synthetic
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 31 - DNA, 3156 bp, Artificial Sequence
  Synthetic
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 32 - DNA, 7009 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctttaattctagtactatgcatgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcagcggaggcaagcgtcctgctgctactaagaaagctggtcaagctaagaaaaagaaaggatcctacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtagcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 33 - protein, 2414 aa, Artificial Sequence
  Synthetic
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 34 - protein, 617 aa, Artificial Sequence
  Synthetic
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 35 - protein, 1497 aa, Artificial Sequence
  Synthetic
RADALDDFDLDMLGSDALDDFDLDMLGSDALDDFDLDMLGSDALDDFDLDMVNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASRADALDDFDLDMLGSDALDDFDLDMLGSDALDDFDLDMLGSDALDDFDLDMLI

SEQ ID NO: 36 - DNA, 4491 bp, Artificial Sequence
  Synthetic
cgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaatc

SEQ ID NO: 37 - protein, 1519 aa, Artificial Sequence
  Synthetic
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 38 - DNA, 4560 bp, Artificial Sequence
  Synthetic
atggactacaaagaccatgacggtgattataaagatcatgacatcgattacaaggatgacgatgacaagatggcccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtttga

SEQ ID NO: 39 - DNA, 86 bp, Artificial Sequence
  Synthetic
gtttaagagctatgctggaaacagcatagcaagtttaaataaggctagtccgttatcaacttgaaaaagtggcaccgagtcggtgc

SEQ ID NO: 40 - RNA, 86 nt, Artificial Sequence
  Synthetic
guuuaagagcuaugcuggaaacagcauagcaaguuuaaauaaggcuaguccguuaucaacuugaaaaaguggcaccgagucggugc

SEQ ID NO: 41 - DNA, 20 bp, Artificial Sequence
  Synthetic
gactagttcgaactatgtgg

SEQ ID NO: 42 - DNA, 20 bp, Artificial Sequence
  Synthetic
gatcatgcagttcacaaatg

SEQ ID NO: 43 - DNA, 20 bp, Artificial Sequence
  Synthetic
gttggccactcatagatacg

SEQ ID NO: 44 - DNA, 20 bp, Artificial Sequence
  Synthetic
ttcctgtcccccgatgaaag

SEQ ID NO: 45 - DNA, 20 bp, Artificial Sequence
  Synthetic
agtacttcacaggtcagagt

SEQ ID NO: 46 - DNA, 20 bp, Artificial Sequence
  Synthetic
atgcctaccaattacaacag

SEQ ID NO: 47 - DNA, 20 bp, Artificial Sequence
  Synthetic
gaacagcttctaagatcggc

SEQ ID NO: 48 - DNA, 20 bp, Artificial Sequence
  Synthetic
ggaacagtctaccatgatat

SEQ ID NO: 49 - DNA, 20 bp, Artificial Sequence
  Synthetic
aagcaataccagtgccacgg

SEQ ID NO: 50 - DNA, 20 bp, Artificial Sequence
  Synthetic
cggccaaagaagagatggag

SEQ ID NO: 51 - DNA, 20 bp, Artificial Sequence
  Synthetic
gccaaaaagagagacccctg

SEQ ID NO: 52 - DNA, 20 bp, Artificial Sequence
  Synthetic
tacctgtagatagtaatgtg

SEQ ID NO: 53 - DNA, 20 bp, Artificial Sequence
  Synthetic
aatgctgggatcagctaccg

SEQ ID NO: 54 - DNA, 20 bp, Artificial Sequence
  Synthetic
cccagagtctgtgaggtcga

SEQ ID NO: 55 - DNA, 20 bp, Artificial Sequence
  Synthetic
ggtgctaaactggtgctctg

SEQ ID NO: 56 - DNA, 20 bp, Artificial Sequence
  Synthetic
tgctgctgatggcgacaatg

SEQ ID NO: 57 - RNA, 20 nt, Artificial Sequence
  Synthetic
gacuaguucgaacuaugugg

SEQ ID NO: 58 - RNA, 20 nt, Artificial Sequence
  Synthetic
gaucaugcaguucacaaaug

SEQ ID NO: 59 - RNA, 20 nt, Artificial Sequence
  Synthetic
guuggccacucauagauacg

SEQ ID NO: 60 - RNA, 20 nt, Artificial Sequence
  Synthetic
uuccugucccccgaugaaag

SEQ ID NO: 61 - RNA, 20 nt, Artificial Sequence
  Synthetic
aguacuucacaggucagagu

SEQ ID NO: 62 - RNA, 20 nt, Artificial Sequence
  Synthetic
augccuaccaauuacaacag

SEQ ID NO: 63 - RNA, 20 nt, Artificial Sequence
  Synthetic
gaacagcuucuaagaucggc

SEQ ID NO: 64 - RNA, 20 nt, Artificial Sequence
  Synthetic
ggaacagucuaccaugauau

SEQ ID NO: 65 - RNA, 20 nt, Artificial Sequence
  Synthetic
aagcaauaccagugccacgg

SEQ ID NO: 66 - RNA, 20 nt, Artificial Sequence
  Synthetic
cggccaaagaagagauggag

SEQ ID NO: 67 - RNA, 20 nt, Artificial Sequence
  Synthetic
gccaaaaagagagaccccug

SEQ ID NO: 68 - RNA, 20 nt, Artificial Sequence
  Synthetic
uaccuguagauaguaaugug

SEQ ID NO: 69 - RNA, 20 nt, Artificial Sequence
  Synthetic
aaugcugggaucagcuaccg

SEQ ID NO: 70 - RNA, 20 nt, Artificial Sequence
  Synthetic
cccagagucugugaggucga

SEQ ID NO: 71 - RNA, 20 nt, Artificial Sequence
  Synthetic
ggugcuaaacuggugcucug

SEQ ID NO: 72 - RNA, 20 nt, Artificial Sequence
  Synthetic
ugcugcugauggcgacaaug